(19) United States
(12) Patent Application Publication
(10) Pub. No.: US 2024/0238514 A1
(43) Pub. Date: Jul. 18, 2024

(54) Sensor Assembly and System for Monitoring Push Medication Delivery to Patients From Syringes

(71) Applicant: Becton, Dickinson and Company, Franklin Lakes, NJ (US)

(72) Inventors: Austin Jason McKinnon, Herriman, UT (US); Erik Kurt Witt, Oakland, NJ (US); Jack Balji, Mahwah, NJ (US)

(21) Appl. No.: 18/414,593

(22) Filed: Jan. 17, 2024

Related U.S. Application Data
(60) Provisional application No. 63/439,689, filed on Jan. 18, 2023.

(51) Int. Cl. A61M 5/172

(52) U.S. Cl. CPC A61M 5/172; A61M 2205/3306; A61M 2205/3389; A61M 2205/581; A61M 2205/583; A61M 2205/6009; A61M 2209/02

(57) ABSTRACT

A sensor assembly may include an image capture device configured to capture one or more images; a user feedback device configured to provide an audible and/or visual output to a user; a mechanical connector configured to connect the sensor assembly to a lumen and/or a syringe configured to be connected to the lumen; and a processor coupled to a memory and configured to: obtain the one or more the images captured by the image capture device; determine, based on the one or more images, information associated with a medication delivery for a patient; and control, based on the information, the user feedback device to provide the audible and/or visual output to the user.

### CROSS-REFERENCE TO RELATED APPLICATION

The present application claims priority to U.S. Provisional Application No. 63/439,689 entitled “Sensor Assembly and System for Monitoring Push Medication Delivery to Patients from Syringes” filed Jan. 18, 2023, the entire disclosure of which is hereby incorporated by reference in its entirety.

### BACKGROUND

### 1. Field

The present disclosure relates generally to medication delivery to patients and, in some non-limiting embodiments or aspects, to a sensory assembly for monitoring push medication delivery to patients from syringes.

### 2. Technical Considerations

Barcoded Medication Administration (BCMA) is an inventory control system that uses barcodes to reduce or prevent human errors in the distribution of prescription medications at hospitals. A goal of BCMA is to make sure that patients are receiving the correct medications at the correct time by electronically validating and documenting medications. The information encoded in barcodes allows for the comparison of the medication being administered with what was ordered for the patient.

Currently, BCMA is the only safety check performed near the patient to reduce or prevent misadministration of medications and aid in documentation in the electronic medical record/electronic health record (EMR/EHR). Accordingly, there is a monitoring gap for drugs delivered via syringe in that an act of connection to a patient's IV line/catheter and dispensing or delivery of a drug may not be monitored.

### SUMMARY

Accordingly, provided are improved systems, devices, products, apparatus, and/or methods for monitoring push medication delivery to patients from syringes.

According to some non-limiting embodiments or aspects, provided is a sensor assembly, including: an image capture device configured to capture one or more images; a user feedback device configured to provide an audible and/or visual output to a user; a mechanical connector configured to connect the sensor assembly to a lumen and/or a syringe configured to be connected to the lumen; and a processor coupled to a memory and configured to: obtain the one or more the images captured by the image capture device; determine, based on the one or more images, information associated with a medication delivery for a patient; and control, based on the information, the user feedback device to provide the audible and/or visual output to the user.

In some non-limiting embodiments or aspects, the image capture device is configured to automatically capture the one or more images in response to a connection of the mechanical connector to the lumen and/or the syringe.

In some non-limiting embodiments or aspects, the sensor assembly further includes: a housing including the image capture device, the user feedback device, and the processor, wherein the mechanical connector extends from a sidewall of the housing, wherein a field-of-view of the image capture device is fixed relative to the housing and/or the mechanical connector, and wherein, with the mechanical connector connecting the sensor assembly to the lumen and/or the syringe connected to the lumen, a field-of-view of the image capture device includes the syringe.

In some non-limiting embodiments or aspects, the mechanical connector includes an opening in a sidewall extending from the housing, and wherein the opening is configured to receive the lumen and/or a barrel of the syringe.

In some non-limiting embodiments or aspects, the image capture device is configured to automatically capture the one or more images in response to the lumen and/or the barrel of the syringe being received in the opening.

In some non-limiting embodiments or aspects, the mechanical connector includes a rigid deformable clip.

In some non-limiting embodiments or aspects, the mechanical connector includes butterfly-style clip.

In some non-limiting embodiments or aspects, the image capture device is configured to automatically capture the one or more images in response to an actuation of the butterfly-style clip.

In some non-limiting embodiments or aspects, the mechanical connector includes arcuate jaws configured to be actuated by one or more buttons, each arcuate jaw connected at a first end to a hinge and including an inwardly facing surface configured to enclose around an exterior surface of the lumen and/or the syringe, the hinge configured to urge second ends of the arcuate jaws together, and the one or more buttons configured to cause the arcuate jaws to separate from each other in response to an actuation of the one or more buttons.

In some non-limiting embodiments or aspects, the image capture device is configured to automatically capture the one or more images in response to the actuation of the one or more buttons.

In some non-limiting embodiments or aspects, the at least one processor is configured to determine, based on the one or more images, the information associated with the medication delivery for the patient by: detecting in the one or more images, a patient identifier element associated with the patient; and determining, based on the detected patient identifier element, patient information associated with the patient, wherein the patient information includes an indication of a type and/or a dosage of a medication to be delivered to the patient and/or a unique identifier of a medication delivery lumen to be used to deliver the medication to the patient.

In some non-limiting embodiments or aspects, the at least one processor is configured to determine, based on the one or more images, the information associated with the medication delivery for the patient by: detecting, in the one or more images, a syringe identifier element associated with the syringe; and determining, based on the detected syringe identifier element, syringe information associated with the syringe, wherein the syringe information includes a type and/or a dosage of a medication contained in the syringe.

In some non-limiting embodiments or aspects, the at least one processor is configured to determine, based on the one or more images, the information associated with the medication delivery for the patient by: determining, based on the patient information and the syringe information, whether the medication contained in the syringe is a correct type and/or a correct dosage of the medication to be delivered to the patient; and controlling, based on whether the medication contained in the syringe includes the correct type and/or the correct dosage of the medication to be delivered to the patient, the user feedback device to provide the audible and/or visual output to the user.

In some non-limiting embodiments or aspects, the at least one processor is configured to determine, based on the one or more images, the information associated with the medication delivery for the patient by: detecting, in the one or more images, a lumen identifier element associated with the lumen; and determining, based on the detected lumen identifier element, lumen information associated with the lumen, wherein the lumen information includes a unique identifier of the lumen.

In some non-limiting embodiments or aspects, the at least one processor is configured to determine, based on the one or more images, the information associated with the medication delivery for the patient by: determining, based on the lumen information and the syringe information, whether the lumen is the medication delivery lumen to be used to deliver the medication to the patient; and controlling, based on whether the lumen includes the medication delivery lumen to be used to deliver the medication to the patient, the user feedback device to provide the audible and/or visual output to the user.

In some non-limiting embodiments or aspects, the one or more images include a series of images including the syringe during the medication delivery for the patient, and wherein the at least one processor is configured to determine, based on the one or more images, the information associated with the medication delivery for the patient by: detecting, in the one or more images, a movement of a stopper and/or a plunger rod of the syringe; determining, based on the detected movement, at least one delivery parameter of the following delivery parameters: a patency of the lumen; an average rate of delivery of the medication; a current rate of the delivery of the medication; a steadiness index of the current rate of delivery of the medication; a volume of the medication delivered; a completion of the medication delivery for the patient; or any combination thereof; and controlling, based on the at least one delivery parameter, the user feedback device to provide the audible and/or visual output to the user.

In some non-limiting embodiments or aspects, the one or more images include a series of images including one or more flush syringes connected to the lumen during a flush procedure, and wherein the least one processor is configured to determine, based on the one or more images, the information associated with the medication delivery for the patient by: detecting, in the one or more images, a lumen identifier element associated with the lumen; determining, based on the detected lumen identifier element, lumen information associated with the lumen, wherein the lumen information includes a unique identifier of the lumen; detecting, in the one or more images, one or more flush syringe identifier elements associated with the one or more flush syringes; determining, based on the one or more detected flush syringe identifier elements, flush syringe information associated with the one or more flush syringes, wherein the flush syringe information includes a number of flush syringes used for the flush procedure; and determining, based on the lumen information and the flush syringe information, that the flush procedure has been performed for the lumen, a number of flush syringes used for the flush procedure, and/or a type of the flush procedure performed (e.g., continuous flushing, partial flushing, pulsatile flushing, etc.).

In some non-limiting embodiments or aspects, the sensor assembly further includes: wireless communications circuitry configured to wirelessly communicate with an external computing device, wherein the at least one processor is configured to control the wireless communication circuitry to wirelessly communicate the information associated with the medication delivery for the patient to the external computing device.

According to some non-limiting embodiments or aspects, provided is a system, including: an external computing device; and one or more sensor assemblies, each sensor assembly of the one or more sensor assemblies including: an image capture device configured to capture one or more images, a user feedback device configured to provide an audible and/or visual output to a user, and a mechanical connector configured to connect the sensor assembly to a lumen and/or a syringe configured to be connected to the lumen; and a wireless communication link configured to facilitate communication between the external computing device, wherein the external computing device includes a processor coupled to a memory and configured to, for each sensor assembly: receive the one or more the images captured by the image capture device; determine, based on the one or more images, information associated with a medication delivery for a patient; and control, based on the information, via the wireless communication link, the user feedback device of the sensor assembly to provide the audible and/or visual output to the user.

In some non-limiting embodiments or aspects, the one or more sensor assemblies include a plurality of sensor assemblies.

According to some non-limiting embodiments or aspects, provided is a method, including: capturing, with an image capture device of a sensor assembly, one or more images, wherein the sensor assembly includes a mechanical connector connecting the sensor assembly to a lumen and/or a syringe configured to be connected to the lumen; obtaining, with at least one processor of the sensor assembly, the one or more the images captured by the image capture device; determining, with the at least one processor of the sensor assembly, based on the one or more images, information associated with a medication delivery for a patient; and controlling, with the at least one processor of the sensor assembly, based on the information, a user feedback device of the sensor assembly to provide an audible and/or visual output to the user.

In some non-limiting embodiments or aspects, the method further includes: automatically capturing, with the image capture device of the sensor assembly, the one or more images in response to a connection of the mechanical connector to the lumen and/or the syringe.

In some non-limiting embodiments or aspects, the sensor assembly further includes a housing including the image capture device, the user feedback device, and the at least one processor, wherein the mechanical connector extends from a sidewall of the housing, wherein a field-of-view of the image capture device is fixed relative to the housing and/or the mechanical connector, and wherein, with the mechanical connector connecting the sensor assembly to the lumen and/or the syringe connected to the lumen, a field-of-view of the image capture device includes the syringe.

In some non-limiting embodiments or aspects, the mechanical connector includes an opening in a sidewall extending from the housing, and wherein the opening is configured to receive the lumen and/or a barrel of the syringe.

In some non-limiting embodiments or aspects, the method further includes: automatically capturing, with the image capture device of the sensor assembly, the one or more images in response to the lumen and/or the barrel of the syringe being received in the opening.

In some non-limiting embodiments or aspects, the mechanical connector includes a rigid deformable clip.

In some non-limiting embodiments or aspects, the mechanical connector includes butterfly-style clip.

In some non-limiting embodiments or aspects, the method further includes: automatically capturing, with the image capture device of the sensor assembly, the one or more images in response to an actuation of the butterfly-style clip.

In some non-limiting embodiments or aspects, the mechanical connector includes arcuate jaws configured to be actuated by one or more buttons, each arcuate jaw connected at a first end to a hinge and including an inwardly facing surface configured to enclose around an exterior surface of the lumen and/or the syringe, the hinge configured to urge second ends of the arcuate jaws together, and the one or more buttons configured to cause the arcuate jaws to separate from each other in response to an actuation of the one or more buttons.

In some non-limiting embodiments or aspects, the method further includes: automatically capturing, with the image capture device of the sensor assembly, the one or more images in response to the actuation of the one or more buttons.

In some non-limiting embodiments or aspects, the determining, with the at least one processor of the senor assembly, based on the one or more images, the information associated with the medication delivery for the patient includes: detecting, in the one or more images, a patient identifier element associated with the patient; and determining, based on the detected patient identifier element, patient information associated with the patient, wherein the patient information includes an indication of a type and/or a dosage of a medication to be delivered to the patient and/or a unique identifier of a medication delivery lumen to be used to deliver the medication to the patient.

In some non-limiting embodiments or aspects, the determining, with the at least one processor of the senor assembly, based on the one or more images, the information associated with the medication delivery for the patient includes: detecting, in the one or more images, a syringe identifier element associated with the syringe; and determining, based on the detected syringe identifier element, syringe information associated with the syringe, wherein the syringe information includes a type and/or a dosage of a medication contained in the syringe.

In some non-limiting embodiments or aspects, the determining, with the at least one processor of the senor assembly, based on the one or more images, the information associated with the medication delivery for the patient includes: determining, based on the patient information and the syringe information, whether the medication contained in the syringe is a correct type and/or a correct dosage of the medication to be delivered to the patient; and controlling, based on whether the medication contained in the syringe includes the correct type and/or the correct dosage of the medication to be delivered to the patient, the user feedback device to provide the audible and/or visual output to the user.

In some non-limiting embodiments or aspects, the determining, with the at least one processor of the senor assembly, based on the one or more images, the information associated with the medication delivery for the patient includes: detecting, in the one or more images, a lumen identifier element associated with the lumen; determining, based on the detected lumen identifier element, lumen information associated with the lumen, wherein the lumen information includes a unique identifier of the lumen.

In some non-limiting embodiments or aspects, the determining, with the at least one processor of the senor assembly, based on the one or more images, the information associated with the medication delivery for the patient includes: determining, based on the lumen information and the syringe information, whether the lumen is the medication delivery lumen to be used to deliver the medication to the patient; and controlling, based on whether the lumen includes the medication delivery lumen to be used to deliver the medication to the patient, the user feedback device to provide the audible and/or visual output to the user.

In some non-limiting embodiments or aspects, the one or more images include a series of images including the syringe during the medication delivery for the patient, and wherein the determining, with the at least one processor of the senor assembly, based on the one or more images, the information associated with the medication delivery for the patient includes: detecting, in the one or more images, a movement of a stopper and/or a plunger rod of the syringe; determining, based on the detected movement, at least one delivery parameter of the following delivery parameters: a patency of the lumen; an average rate of delivery of the medication; a current rate of the delivery of the medication; a steadiness index of the current rate of delivery of the medication; a volume of the medication delivered; a completion of the medication delivery for the patient; or any combination thereof; and controlling, based on the at least one delivery parameter, the user feedback device to provide the audible and/or visual output to the user.

In some non-limiting embodiments or aspects, the one or more images include a series of images including one or more flush syringes connected to the lumen during a flush procedure, and wherein the determining, with the at least one processor of the senor assembly, based on the one or more images, the information associated with the medication delivery for the patient includes: detecting, in the one or more images, a lumen identifier element associated with the lumen; determining, based on the detected lumen identifier element, lumen information associated with the lumen, wherein the lumen information includes a unique identifier of the lumen; detecting, in the one or more images, one or more flush syringe identifier elements associated with the one or more flush syringes; determining, based on the one or more detected flush syringe identifier elements, flush syringe information associated with the one or more flush syringes, wherein the flush syringe information includes a number of flush syringes used for the flush procedure; and determining, based on the lumen information and the flush syringe information, that the flush procedure has been performed for the lumen, a number of flush syringes used for the flush procedure, and/or a type of the flush procedure performed (e.g., continuous flushing, partial flushing, pulsatile flushing, etc.).

In some non-limiting embodiments or aspects, the method further includes: controlling, with the at least one processor of the sensor assembly, wireless communications circuitry of the sensor assembly to wirelessly communicate the information associated with the medication delivery for the patient to an external computing device.

Further embodiments or aspects are set forth in the following numbered clauses:

Clause 1. A sensor assembly, comprising: an image capture device configured to capture one or more images; a user feedback device configured to provide an audible and/or visual output to a user; a mechanical connector configured to connect the sensor assembly to a lumen and/or a syringe configured to be connected to the lumen; and a processor coupled to a memory and configured to: obtain the one or more the images captured by the image capture device; determine, based on the one or more images, information associated with a medication delivery for a patient; and control, based on the information, the user feedback device to provide the audible and/or visual output to the user.

Clause 2. The sensor assembly of clause 1, wherein the image capture device is configured to automatically capture the one or more images in response to a connection of the mechanical connector to the lumen and/or the syringe.

Clause 3. The sensor assembly of any of clause 1 and clause 2, further comprising: a housing including the image capture device, the user feedback device, and the processor, wherein the mechanical connector extends from a sidewall of the housing, wherein a field-of-view of the image capture device is fixed relative to the housing and/or the mechanical connector, and wherein, with the mechanical connector connecting the sensor assembly to the lumen and/or the syringe connected to the lumen, a field-of-view of the image capture device includes the syringe.

Clause 4. The sensor assembly of any of clauses 1-3, wherein the mechanical connector includes an opening in a sidewall extending from the housing, and wherein the opening is configured to receive the lumen and/or a barrel of the syringe.

Clause 5. The sensor assembly of any of clauses 1-4, wherein the image capture device is configured to automatically capture the one or more images in response to the lumen and/or the barrel of the syringe being received in the opening.

Clause 6. The sensor assembly of any of clauses 1-5, wherein the mechanical connector includes a rigid deformable clip.

Clause 7. The sensor assembly of any of clauses 1-6, wherein the mechanical connector includes butterfly-style clip.

Clause 8. The sensor assembly of any of clauses 1-7, wherein the image capture device is configured to automatically capture the one or more images in response to an actuation of the butterfly-style clip.

Clause 9. The sensor assembly of any of clauses 1-8, wherein the mechanical connector includes arcuate jaws configured to be actuated by one or more buttons, each arcuate jaw connected at a first end to a hinge and including an inwardly facing surface configured to enclose around an exterior surface of the lumen and/or the syringe, the hinge configured to urge second ends of the arcuate jaws together, and the one or more buttons configured to cause the arcuate jaws to separate from each other in response to an actuation of the one or more buttons.

Clause 10. The sensor assembly of any of clauses 1-9, wherein the image capture device is configured to automatically capture the one or more images in response to the actuation of the one or more buttons.

Clause 11. The sensor assembly of any of clauses 1-10, wherein the at least one processor is configured to determine, based on the one or more images, the information associated with the medication delivery for the patient by: detecting in the one or more images, a patient identifier element associated with the patient; and determining, based on the detected patient identifier element, patient information associated with the patient, wherein the patient information includes an indication of a type and/or a dosage of a medication to be delivered to the patient and/or a unique identifier of a medication delivery lumen to be used to deliver the medication to the patient.

Clause 12. The sensor assembly of any of clauses 1-11, wherein the at least one processor is configured to determine, based on the one or more images, the information associated with the medication delivery for the patient by: detecting, in the one or more images, a syringe identifier element associated with the syringe; and determining, based on the detected syringe identifier element, syringe information associated with the syringe, wherein the syringe information includes a type and/or a dosage of a medication contained in the syringe.

Clause 13. The sensor assembly of any of clauses 1-12, wherein the at least one processor is configured to determine, based on the one or more images, the information associated with the medication delivery for the patient by: determining, based on the patient information and the syringe information, whether the medication contained in the syringe is a correct type and/or a correct dosage of the medication to be delivered to the patient; and controlling, based on whether the medication contained in the syringe includes the correct type and/or the correct dosage of the medication to be delivered to the patient, the user feedback device to provide the audible and/or visual output to the user.

Clause 14. The sensor assembly of any of clauses 1-13, wherein the at least one processor is configured to determine, based on the one or more images, the information associated with the medication delivery for the patient by: detecting, in the one or more images, a lumen identifier element associated with the lumen; determining, based on the detected lumen identifier element, lumen information associated with the lumen, wherein the lumen information includes a unique identifier of the lumen.

Clause 15. The sensor assembly of any of clauses 1-14, wherein the at least one processor is configured to determine, based on the one or more images, the information associated with the medication delivery for the patient by: determining, based on the lumen information and the syringe information, whether the lumen is the medication delivery lumen to be used to deliver the medication to the patient; and controlling, based on whether the lumen includes the medication delivery lumen to be used to deliver the medication to the patient, the user feedback device to provide the audible and/or visual output to the user.

Clause 16. The sensor assembly of any of clauses 1-15, wherein the one or more images include a series of images including the syringe during the medication delivery for the patient, and wherein the at least one processor is configured to determine, based on the one or more images, the information associated with the medication delivery for the patient by: detecting, in the one or more images, a movement of a stopper and/or a plunger rod of the syringe; determining, based on the detected movement, at least one delivery parameter of the following delivery parameters: a patency of the lumen; an average rate of delivery of the medication; a current rate of the delivery of the medication; a steadiness index of the current rate of delivery of the medication; a volume of the medication delivered; a completion of the medication delivery for the patient; or any combination thereof; and controlling, based on the at least one delivery parameter, the user feedback device to provide the audible and/or visual output to the user.

Clause 17. The sensor assembly of any of clauses 1-16, wherein the one or more images include a series of images including one or more flush syringes connected to the lumen during a flush procedure, and wherein the least one processor is configured to determine, based on the one or more images, the information associated with the medication delivery for the patient by: detecting, in the one or more images, a lumen identifier element associated with the lumen; determining, based on the detected lumen identifier element, lumen information associated with the lumen, wherein the lumen information includes a unique identifier of the lumen; detecting, in the one or more images, one or more flush syringe identifier elements associated with the one or more flush syringes; determining, based on the one or more detected flush syringe identifier elements, flush syringe information associated with the one or more flush syringes, wherein the flush syringe information includes a number of flush syringes used for the flush procedure; and determining, based on the lumen information and the flush syringe information, that the flush procedure has been performed for the lumen, a number of flush syringes used for the flush procedure, and/or a type of the flush procedure performed (e.g., continuous flushing, partial flushing, pulsatile flushing, etc.).

Clause 18. The sensor assembly of any of clauses 1-17, further comprising: wireless communications circuitry configured to wirelessly communicate with an external computing device, wherein the at least one processor is configured to control the wireless communication circuitry to wirelessly communicate the information associated with the medication delivery for the patient to the external computing device.

Clause 19. A system, comprising: an external computing device; and one or more sensor assemblies, each sensor assembly of the one or more sensor assemblies including: an image capture device configured to capture one or more images, a user feedback device configured to provide an audible and/or visual output to a user, and a mechanical connector configured to connect the sensor assembly to a lumen and/or a syringe configured to be connected to the lumen; and a wireless communication link configured to facilitate communication between the external computing device, wherein the external computing device includes a processor coupled to a memory and configured to, for each sensor assembly: receive the one or more the images captured by the image capture device; determine, based on the one or more images, information associated with a medication delivery for a patient; and control, based on the information, via the wireless communication link, the user feedback device of the sensor assembly to provide the audible and/or visual output to the user.

Clause 20. The system of clause 19, wherein the one or more sensor assemblies include a plurality of sensor assemblies.

Clause 21. A method, comprising: capturing, with an image capture device of a sensor assembly, one or more images, wherein the sensor assembly includes a mechanical connector connecting the sensor assembly to a lumen and/or a syringe configured to be connected to the lumen; obtaining, with at least one processor of the sensor assembly, the one or more the images captured by the image capture device; determining, with the at least one processor of the sensor assembly, based on the one or more images, information associated with a medication delivery for a patient; and controlling, with the at least one processor of the sensor assembly, based on the information, a user feedback device of the sensor assembly to provide an audible and/or visual output to the user.

Clause 22. The method of clause 21, further comprising: automatically capturing, with the image capture device of the sensor assembly, the one or more images in response to a connection of the mechanical connector to the lumen and/or the syringe.

Clause 23. The method of any of clause 21 and clause 22, wherein the sensor assembly further includes a housing including the image capture device, the user feedback device, and the at least one processor, wherein the mechanical connector extends from a sidewall of the housing, wherein a field-of-view of the image capture device is fixed relative to the housing and/or the mechanical connector, and wherein, with the mechanical connector connecting the sensor assembly to the lumen and/or the syringe connected to the lumen, a field-of-view of the image capture device includes the syringe.

Clause 24. The method of any of clauses 21-23, wherein the mechanical connector includes an opening in a sidewall extending from the housing, and wherein the opening is configured to receive the lumen and/or a barrel of the syringe.

Clause 25. The method of any of clauses 21-24, further comprising: automatically capturing, with the image capture device of the sensor assembly, the one or more images in response to the lumen and/or the barrel of the syringe being received in the opening.

Clause 26. The method of any of clauses 21-25, wherein the mechanical connector includes a rigid deformable clip.

Clause 27. The method of any of clauses 21-26, wherein the mechanical connector includes butterfly-style clip.

Clause 28. The method of any of clauses 21-27, further comprising: automatically capturing, with the image capture device of the sensor assembly, the one or more images in response to an actuation of the butterfly-style clip.

Clause 29. The method of any of clauses 21-28, wherein the mechanical connector includes arcuate jaws configured to be actuated by one or more buttons, each arcuate jaw connected at a first end to a hinge and including an inwardly facing surface configured to enclose around an exterior surface of the lumen and/or the syringe, the hinge configured to urge second ends of the arcuate jaws together, and the one or more buttons configured to cause the arcuate jaws to separate from each other in response to an actuation of the one or more buttons.

Clause 30. The method of any of clauses 21-29, further comprising: automatically capturing, with the image capture device of the sensor assembly, the one or more images in response to the actuation of the one or more buttons.

Clause 31. The method of any of clauses 21-30, wherein the determining, with the at least one processor of the senor assembly, based on the one or more images, the information associated with the medication delivery for the patient includes: detecting, in the one or more images, a patient identifier element associated with the patient; and determining, based on the detected patient identifier element, patient information associated with the patient, wherein the patient information includes an indication of a type and/or a dosage of a medication to be delivered to the patient and/or a unique identifier of a medication delivery lumen to be used to deliver the medication to the patient.

Clause 32. The method of any of clauses 21-31, wherein the determining, with the at least one processor of the senor assembly, based on the one or more images, the information associated with the medication delivery for the patient includes: detecting, in the one or more images, a syringe identifier element associated with the syringe; and determining, based on the detected syringe identifier element, syringe information associated with the syringe, wherein the syringe information includes a type and/or a dosage of a medication contained in the syringe.

Clause 33. The method of any of clauses 21-32, wherein the determining, with the at least one processor of the senor assembly, based on the one or more images, the information associated with the medication delivery for the patient includes: determining, based on the patient information and the syringe information, whether the medication contained in the syringe is a correct type and/or a correct dosage of the medication to be delivered to the patient; and controlling, based on whether the medication contained in the syringe includes the correct type and/or the correct dosage of the medication to be delivered to the patient, the user feedback device to provide the audible and/or visual output to the user.

Clause 34. The method of any of clauses 21-33, wherein the determining, with the at least one processor of the senor assembly, based on the one or more images, the information associated with the medication delivery for the patient includes: detecting, in the one or more images, a lumen identifier element associated with the lumen; determining, based on the detected lumen identifier element, lumen information associated with the lumen, wherein the lumen information includes a unique identifier of the lumen.

Clause 35. The method of any of clauses 21-34, wherein the determining, with the at least one processor of the senor assembly, based on the one or more images, the information associated with the medication delivery for the patient includes: determining, based on the lumen information and the syringe information, whether the lumen is the medication delivery lumen to be used to deliver the medication to the patient; and controlling, based on whether the lumen includes the medication delivery lumen to be used to deliver the medication to the patient, the user feedback device to provide the audible and/or visual output to the user.

Clause 36. The method of any of clauses 21-35, wherein the one or more images include a series of images including the syringe during the medication delivery for the patient, and wherein the determining, with the at least one processor of the senor assembly, based on the one or more images, the information associated with the medication delivery for the patient includes: detecting, in the one or more images, a movement of a stopper and/or a plunger rod of the syringe; determining, based on the detected movement, at least one delivery parameter of the following delivery parameters: a patency of the lumen; an average rate of delivery of the medication; a current rate of the delivery of the medication; a steadiness index of the current rate of delivery of the medication; a volume of the medication delivered; a completion of the medication delivery for the patient; or any combination thereof; and controlling, based on the at least one delivery parameter, the user feedback device to provide the audible and/or visual output to the user.

Clause 37. The method of any of clauses 21-36, wherein the one or more images include a series of images including one or more flush syringes connected to the lumen during a flush procedure, and wherein the determining, with the at least one processor of the senor assembly, based on the one or more images, the information associated with the medication delivery for the patient includes: detecting, in the one or more images, a lumen identifier element associated with the lumen; determining, based on the detected lumen identifier element, lumen information associated with the lumen, wherein the lumen information includes a unique identifier of the lumen; detecting, in the one or more images, one or more flush syringe identifier elements associated with the one or more flush syringes; determining, based on the one or more detected flush syringe identifier elements, flush syringe information associated with the one or more flush syringes, wherein the flush syringe information includes a number of flush syringes used for the flush procedure; and determining, based on the lumen information and the flush syringe information, that the flush procedure has been performed for the lumen, a number of flush syringes used for the flush procedure, and/or a type of the flush procedure performed (e.g., continuous flushing, partial flushing, pulsatile flushing, etc.).

Clause 38. The method of any of clauses 21-37, further comprising: controlling, with the at least one processor of the sensor assembly, wireless communications circuitry of the sensor assembly to wirelessly communicate the information associated with the medication delivery for the patient to an external computing device.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of limits. As used in the specification and the claims, the singular form of “a,” “an,” and “the” include plural referents unless the context clearly dictates otherwise.

### BRIEF DESCRIPTION OF THE DRAWINGS

Additional advantages and details of embodiments or aspects of the present disclosure are explained in greater detail below with reference to the exemplary embodiments that are illustrated in the accompanying schematic figures, in which:

FIG. 1A is a diagram of non-limiting embodiments or aspects of an environment in which systems, devices, products, apparatus, and/or methods, described herein, may be implemented;

FIG. 1B is a diagram of non-limiting embodiments or aspects of components of one or more devices and/or one or more systems of FIG. 1A;

FIGS. 2A-2E illustrate an implementation of non-limiting embodiments or aspects of a sensor assembly;

FIGS. 3A-3D illustrate an implementation of non-limiting embodiments or aspects of a base for a sensor assembly;

FIGS. 4A-4H illustrate an implementation of non-limiting embodiments or aspects of a sensor assembly;

FIGS. 5A-5H illustrate an implementation of non-limiting embodiments or aspects of a sensor assembly;

FIG. 6 illustrates an implementation of non-limiting embodiments or aspects of a communication network for a sensor assembly;

FIG. 7 is a flowchart of non-limiting embodiments or aspects of a process for monitoring push medication delivery to patients from syringes;

FIGS. 8A and 8B are a flowchart of non-limiting embodiments or aspects of a process for monitoring push medication delivery to patients from syringes;

FIG. 9 is a flowchart of non-limiting embodiments or aspects of a process for monitoring push medication delivery to patients from syringes; and

FIG. 10 is a flowchart of non-limiting embodiments or aspects of a process for monitoring push medication delivery to patients from syringes.

### DETAILED DESCRIPTION

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting.

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the present disclosure as it is oriented in the drawing figures. However, it is to be understood that the present disclosure may assume various alternative variations and step sequences, except where expressly specified to the contrary. It is also to be understood that the specific devices and processes illustrated in the attached drawings, and described in the following specification, are simply exemplary embodiments or aspects of the present disclosure. Hence, specific dimensions and other physical characteristics related to the embodiments or aspects of the embodiments disclosed herein are not to be considered as limiting unless otherwise indicated.

No aspect, component, element, structure, act, step, function, instruction, and/or the like used herein should be construed as critical or essential unless explicitly described as such. Also, as used herein, the articles “a” and “an” are intended to include one or more items, and may be used interchangeably with “one or more” and “at least one.” Furthermore, as used herein, the term “set” is intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least in partially on” unless explicitly stated otherwise.

As used herein, the terms “communication” and “communicate” refer to the receipt or transfer of one or more signals, messages, commands, or other type of data. For one unit (e.g., any device, system, or component thereof) to be in communication with another unit means that the one unit is able to directly or indirectly receive data from and/or transmit data to the other unit. This may refer to a direct or indirect connection that is wired and/or wireless in nature. Additionally, two units may be in communication with each other even though the data transmitted may be modified, processed, relayed, and/or routed between the first and second unit. For example, a first unit may be in communication with a second unit even though the first unit passively receives data and does not actively transmit data to the second unit. As another example, a first unit may be in communication with a second unit if an intermediary unit processes data from one unit and transmits processed data to the second unit. It will be appreciated that numerous other arrangements are possible.

It will be apparent that systems and/or methods, described herein, can be implemented in different forms of hardware, software, or a combination of hardware and software. The actual specialized control hardware or software code used to implement these systems and/or methods is not limiting of the implementations. Thus, the operation and behavior of the systems and/or methods are described herein without reference to specific software code, it being understood that software and hardware can be designed to implement the systems and/or methods based on the description herein.

Some non-limiting embodiments or aspects are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

As used herein, the term “computing device” or “computer device” may refer to one or more electronic devices that are configured to directly or indirectly communicate with or over one or more networks. The computing device may be a mobile device, a desktop computer, or the like. Furthermore, the term “computer” may refer to any computing device that includes the necessary components to receive, process, and output data, and normally includes a display, a processor, a memory, an input device, and a network interface. An “application” or “application program interface” (API) refers to computer code or other data sorted on a computer-readable medium that may be executed by a processor to facilitate the interaction between software components, such as a client-side front-end and/or server-side back-end for receiving data from the client. An “interface” refers to a generated display, such as one or more graphical user interfaces (GUIs) with which a user may interact, either directly or indirectly (e.g., through a keyboard, mouse, touchscreen, etc.).

As used herein, the term “server” may refer to or include one or more processors or computers, storage devices, or similar computer arrangements that are operated by or facilitate communication and processing for multiple parties in a network environment, such as the Internet, although it will be appreciated that communication may be facilitated over one or more public or private network environments and that various other arrangements are possible. Further, multiple computers, e.g., servers, or other computerized devices, such as POS devices, directly or indirectly communicating in the network environment may constitute a “system,” such as a merchant's POS system. As used herein, the term “data center” may include one or more servers, or other computing devices, and/or databases.

As used herein, the term “mobile device” may refer to one or more portable electronic devices configured to communicate with one or more networks. As an example, a mobile device may include a cellular phone (e.g., a smartphone or standard cellular phone), a portable computer (e.g., a tablet computer, a laptop computer, etc.), a wearable device (e.g., a watch, pair of glasses, lens, clothing, and/or the like), a personal digital assistant (PDA), and/or other like devices. The terms “client device” and “user device,” as used herein, refer to any electronic device that is configured to communicate with one or more servers or remote devices and/or systems. A client device or user device may include a mobile device, a network-enabled appliance (e.g., a network-enabled television, refrigerator, thermostat, and/or the like), a computer, and/or any other device or system capable of communicating with a network.

As used herein, the term “application” or “application program interface” (API) refers to computer code, a set of rules, or other data sorted on a computer-readable medium that may be executed by a processor to facilitate interaction between software components, such as a client-side front-end and/or server-side back-end for receiving data from the client. An “interface” refers to a generated display, such as one or more graphical user interfaces (GUIs) with which a user may interact, either directly or indirectly (e.g., through a keyboard, mouse, etc.).

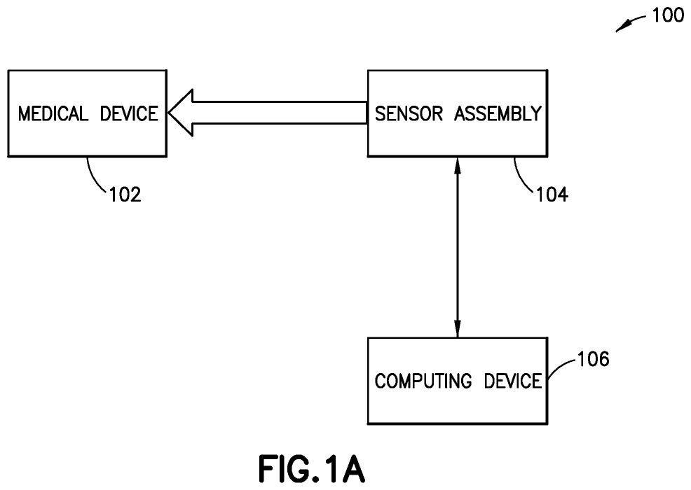

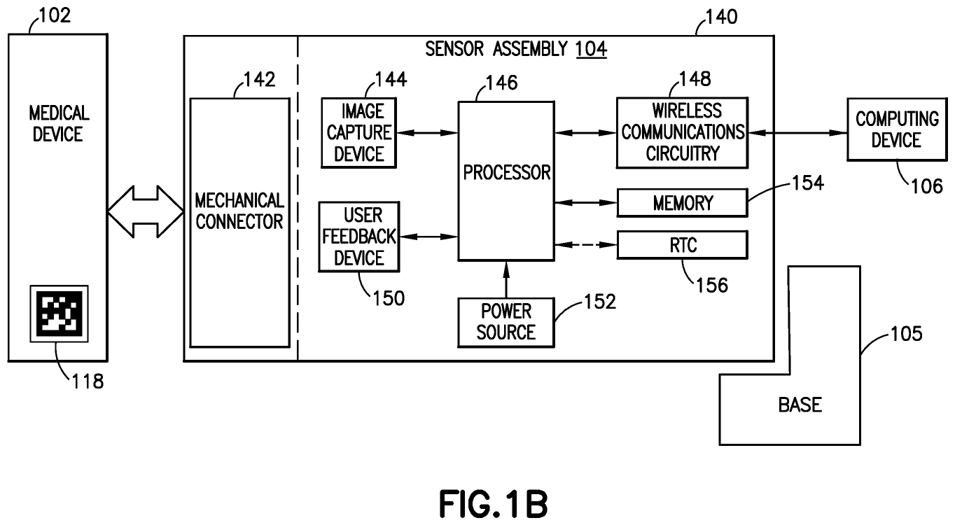

Referring to FIG. 1A, non-limiting embodiments or aspects of an environment 100 in which systems, devices, products, apparatus, and/or methods, as described herein, may be implemented is shown. As shown in FIG. 1A, environment 100 may include medical device 102, sensor assembly 104, and/or computing device 106. Referring also to FIG. 1B, non-limiting embodiments or aspects of components of one or more devices and/or one or more systems of FIG. 1A is shown.

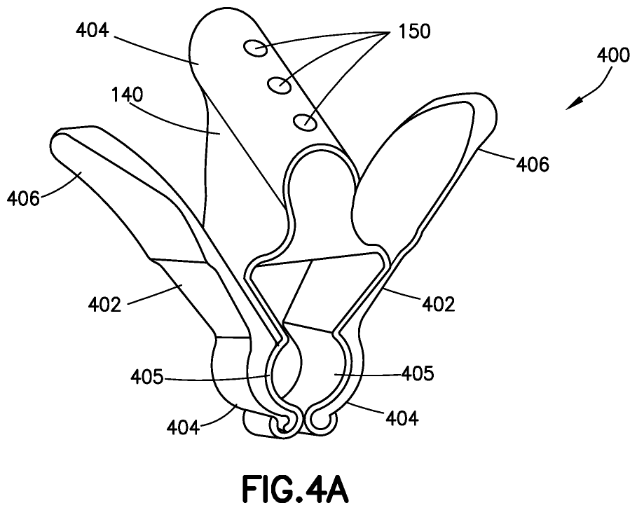

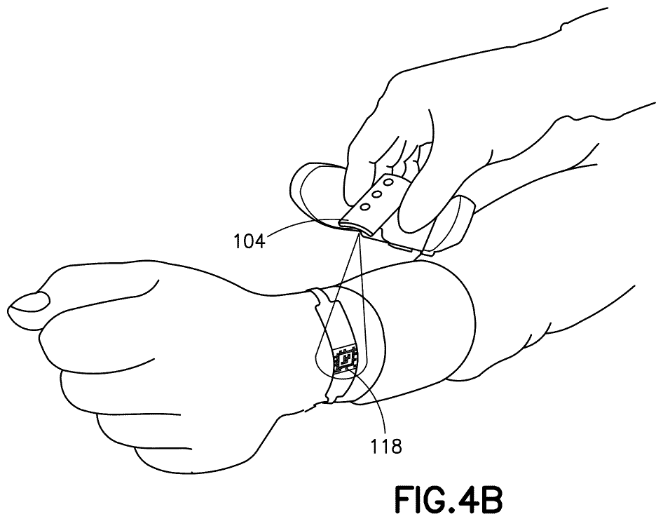

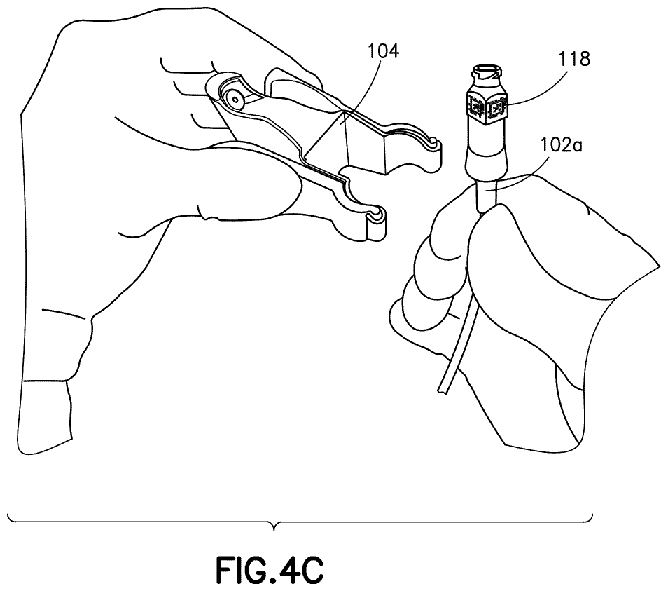

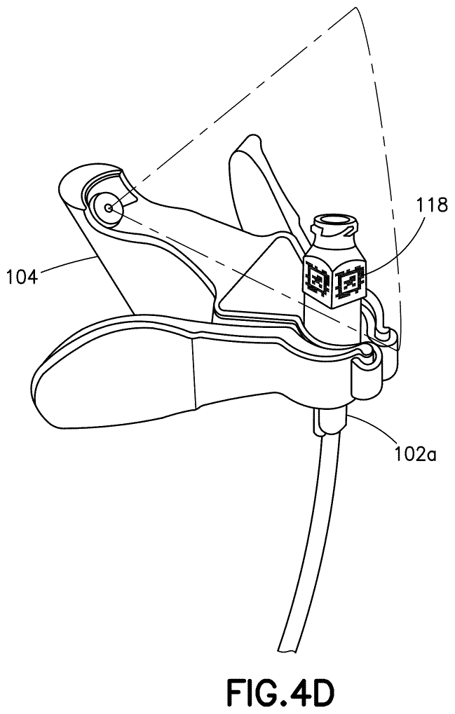

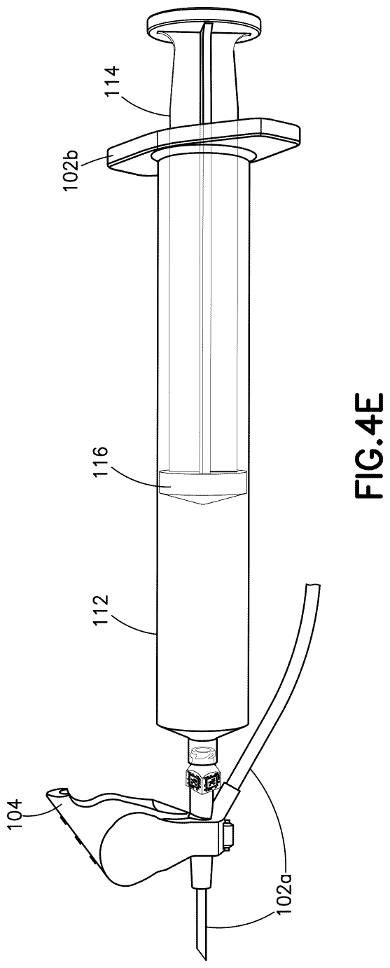

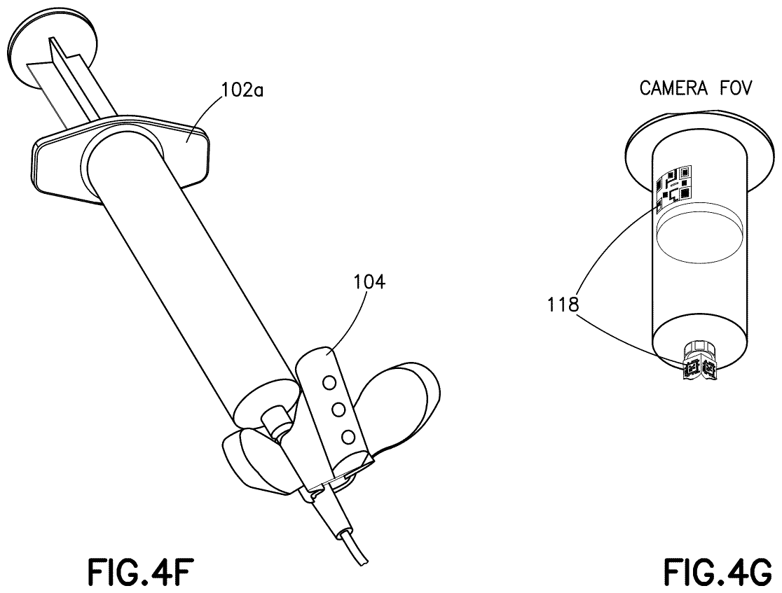

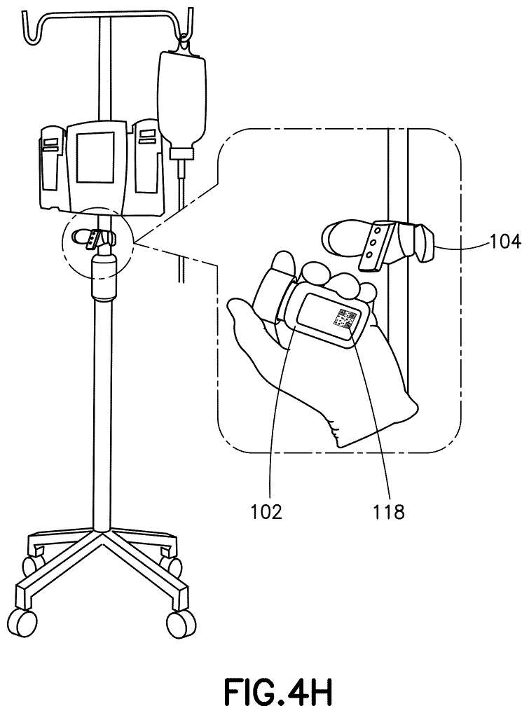

Medical device 102 may include a lumen 102a (e.g., an IV line, a catheter, etc.) and/or a syringe 102b configured to be connected to the lumen 102a (e.g., via a needless connector, etc.). Syringe 102b may include syringe barrel 112, plunger rod 114, and/or stopper 116 (see, e.g., FIG. 4E). Syringe barrel 112 may extend between a proximal end including a proximal opening configured to receive stopper 116 and/or plunger rod 114 and a distal end including a distal opening (e.g., a luer lock opening, etc.) configured to be connected to an IV line or catheter (e.g., via a needles connector, etc.) and through which a fluid (e.g. a medication, etc.) can be expelled from syringe 102b. In some non-limiting embodiments or aspects, medical device 102 may include at least one of the following medical devices: a peripheral IV catheter (PIVC), a peripherally inserted central catheter (PICC), a midline catheter, a central venous catheter (CVC), a needleless connector, a catheter dressing, a catheter stabilization device, a disinfectant cap, a disinfectant swab or wipe, an IV tubing set, an extension set, a Y connector, a stopcock, an infusion pump, a flush syringe, a medication delivery syringe, a caregiver glove, an IV fluid bag, a medication dispensing cabinet, an ultrasound device, a sharps collector, or any combination thereof.

Identifier element 118 (e.g., a tag, a label, a code, etc.) may be associated with (e.g., removably attached to, permanently attached to, integrated with, implemented on, etc.) medical device 102 (e.g., lumen 102a, syringe 102b, etc.) and/or a patient (e.g., a patient wristband, etc.). For example, identifier element 118 may encapsulate a device identifier associated with a type and/or contents of medical device 102 associated with identifier element 118 and/or uniquely identify medical device 102 associated with identifier element 118 from other medical devices and/or indicate an orientation of the medical device 102 within environment 100 and/or with respect to another medical device (e.g., a fluid flow path direction through a medical device, an input or inlet position and an output or outlet position of a medical device, etc.). As an example, identifier element 118 may encapsulate a unique patient identifier associated with a specific patient and/or patient information, such as, the unique patient identification number, drug allergies, blood type, patient vital signs, lab results, current disease states and/or clinical diagnoses, drugs previously administered, medical orders (e.g., medication, dose, route of administration, treatment schedule, etc.), historic patient information (e.g., disease state, clinical diagnosis, dosing history, etc.), and/or the like. Identifier element 118 may include at least one of the following: a colored pattern, a reflective pattern, a fluorescent pattern, a predetermined shape and color, a LED pattern, a barcode (e.g., a 1D barcode, a 2D barcode, etc.), a fiducial marker (e.g., an AprilTag, etc.), a hologram marker, or any combination thereof, which may encapsulate the device identifier and/or patient identifier.

Sensor assembly 104 may include housing 140 and mechanical connector 142. Housing 140 may include (e.g., house, encompass, etc.) image capture device 144, processor 146, wireless communications circuitry 148, user feedback device 150, power source 152, memory 154, and/or real-time clock (RTC) 156.

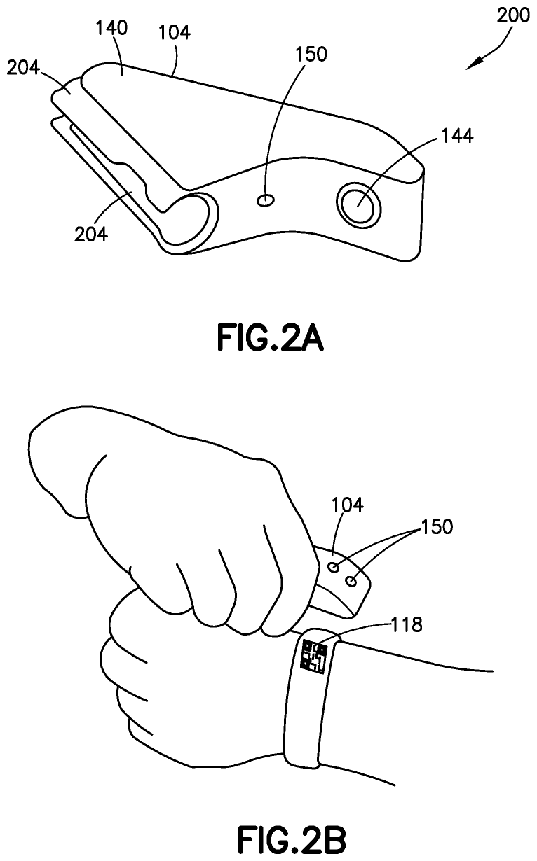

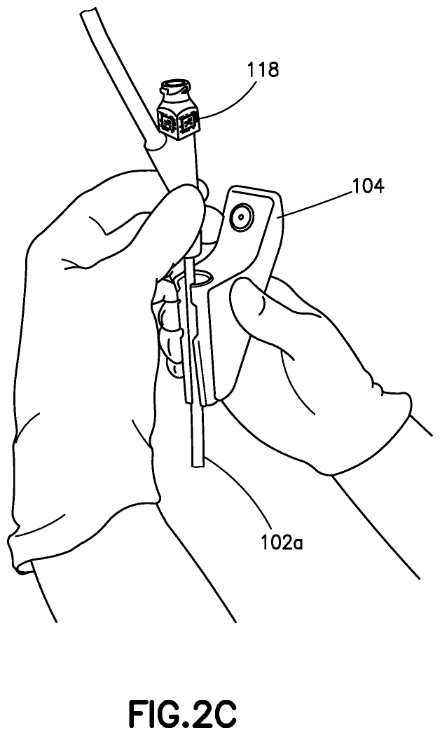

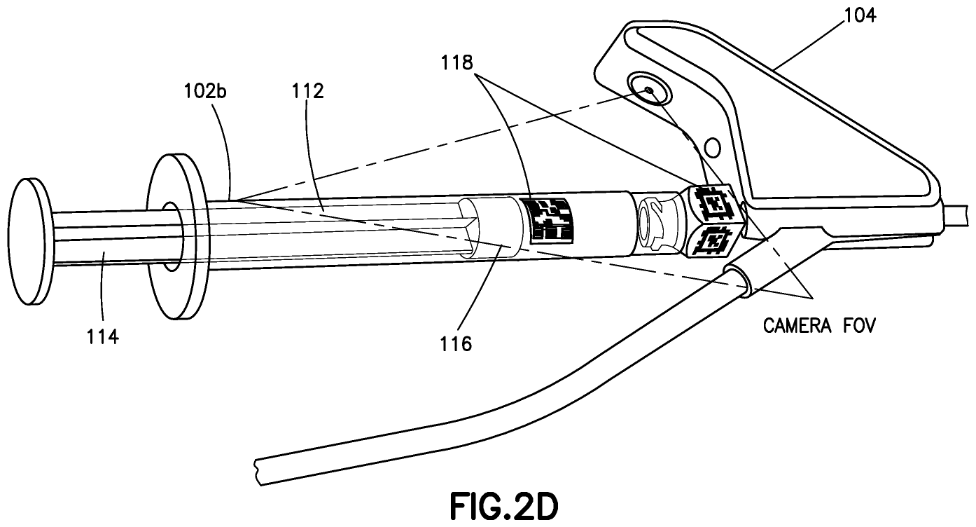

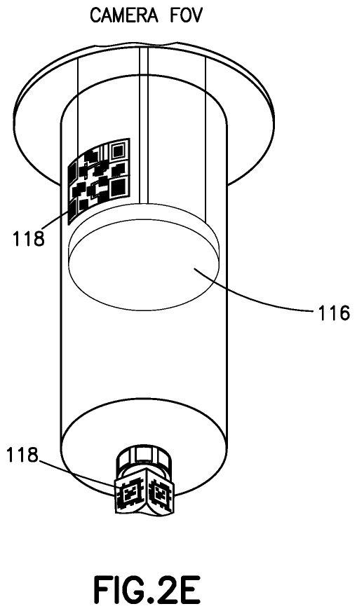

Mechanical connector 142 may be configured to connect or attach (e.g., removably attach, etc.) housing 140 to an exterior surface of medical device 102 (e.g., to lumen 102a, to barrel 112 of syringe 102b, etc.). For example, housing 140 may be removably attached to mechanical connector 142, permanently attached to mechanical connector 142, and/or integrated with mechanical connector 142. As an example mechanical connector 142 may extend from a sidewall of housing 140. Further details regarding non-limiting embodiments or aspects of mechanical connector 142 are provided below with regard to FIGS. 2A-2E, 3A-3D, 4A-4H, and 5A-5H.

Image capture device 144 may include a camera, such as a digital monocular camera, an infrared camera, a stereo camera, a single camera, and/or the like, configured to capture one or more images. For example, image capture device 144 may be configured to capture a plurality of images over a period of time (e.g., as a series of images, etc.). In some non-limiting embodiment or aspects, image capture device is configured to automatically capture the one or more images in response to a connection of mechanical connector 142 to lumen 102a and/or syringe 102b. In some non-limiting embodiments or aspects, a field-of-view of image capture device 144 is fixed relative to housing 140 and/or mechanical connector 142 such that, with mechanical connector 142 connecting housing 140 of sensor assembly 104 to lumen 102a and/or syringe 102b connected to lumen 102a, the field-of-view of image capture device includes lumen 102a and/or syringe 102b. For example, the fixed field-of-view of image capture device 144 may include identifier elements 118 on lumen 102a and/or syringe 102b.

Processor 146 (e.g., a central processing unit (CPU), a graphics processing unit (GPU), etc.) may be programmed and/or configured to obtain the one or more the images captured by image capture device 142; determine, based on the one or more images, information associated with a medication delivery for a patient; and control, based on the information, user feedback device 150 to provide an audible and/or visual output to a user. In some non-limiting embodiments or aspects, processor 146 may include a low power microcontroller unit (MCU). For example, information associated with a medication delivery for a patient may include patient information, lumen information, syringe information, flush syringe information, and/or the like.

Processor 146 may be programmed and/or configured to process the one or more images using one or more object detection techniques (e.g., a deep learning technique, an image processing technique, an image segmentation technique, etc.) to identify identifier element 118, to determine whether syringe 102b is connected to lumen 102a, and/or to detect movement of an actuation mechanism or fluid expulsion mechanism, such as a plunger rod 114 and/or stopper 116, during a fluid delivery procedure using syringe 102b. For example, a deep learning technique may include a bounding box technique that generates a box label for objects (e.g., identifier element 118, stopper 116, plunger rod 114, etc.) of interest in images, an image masking technique (e.g., masked FRCNN (RCNN or CNN) that captures specific shapes of objects (e.g., identifier element 118, stopper 116, plunger rod 114, etc.) in images, a trained neural network that identifies objects (e.g., identifier element 118, stopper 116, plunger rod 114, etc.) in images, and/or the like. As an example, an image processing technique may include a cross correlation image processing technique, an image contrasting technique, a binary or colored filtering technique, and/or the like.

In some non-limiting embodiments or aspects, identifier element 118 may include an AprilTag. For example, identifier element 118 may include an AprilTag V3 of type customTag 48h12, which enables using AprilTag V3 detection to determine a unique ID, which may indicate a type of medical device 102 associated with identifier element 118 (e.g., in leading digits, etc.), a unique serial number for that specific medical device 102 (e.g., in the trailing digits, etc.), and/or a location (e.g., x, y, and z coordinates, directional vectors for Z, Y, and X axes, etc.) of identifier element 118 in a field-of-view (FOV) of an image capture device 144. However, non-limiting embodiments or aspects are not limited thereto, and identifier element 118 may include a QR code, a barcode (e.g., a 1D barcode, a 2D barcode, etc.), an Aztec code, a Data Matrix code, an ArUco marker, a colored pattern, a reflective pattern, a fluorescent pattern, a predetermined shape and/or color (e.g., a red pentagon, a blue hexagon, etc.), an LED pattern, a hologram, and/or the like that encapsulates an identifier associated with a type of medical device 102 associated with identifier element 118, uniquely identifies medical device 102 associated with identifier element 118 from other medical devices, and/or encapsulates pose information associated with a 3D position of identifier element 118.

Wireless communications circuitry 148 may be configured to wirelessly communicate with computing device 106. For example, wireless communications circuitry 148 may be configured to communicate information associated with a medication delivery for a patient to computing device 106. In some non-limiting embodiments or aspects, wireless communication device 148 includes one or more computing devices, chips, contactless transmitters, contactless transceivers, NFC transmitters, RFID transmitters, contact based transmitters, Bluetooth transceivers® and/or the like that enables wireless communications circuitry 148 to receive information directly from and/or communicate information directly to computing device 106 via a short range wireless communication connection (e.g., a communication connection that uses NFC protocol, a communication connection that uses Radio-frequency identification (RFID), a communication connection that uses a Bluetooth® wireless technology standard, and/or the like).

User feedback device 150 may be configured to provide audible and/or visual output to a user. For example, user feedback device 150 may include at least one of the following: a display (e.g., an LCD display, etc.), a light-emitting diode (LED), a plurality of LEDs, an audio output device (e.g., a buzzer, a speaker, etc.), a tactile or haptic feedback device (e.g., a vibrator, etc.), or any combination thereof.

Power source 152 may be configured to power image capture device 144, processor 146, wireless communications circuitry 148, user feedback device 150, memory 154, and/or RTC 156. For example, power source 152 may include a battery (e.g., a rechargeable battery, a disposable battery, etc.), an energy harvester (e.g., an energy harvester configured to derive energy from one or more external sources, such as electromagnetic energy, solar energy, thermal energy, wind energy, salinity gradients, kinetic energy, and/or the like, etc.), or any combination thereof.

Memory 154 may include random access memory (RAM), read-only memory (ROM), and/or another type of dynamic or static storage device (e.g., flash memory, magnetic memory, optical memory, etc.) that stores information and/or instructions for use by processor 146. For example, sensor assembly 104 may perform one or more processes described herein based on processor 146 executing software instructions stored by a computer-readable medium, such as memory 154. A computer-readable medium (e.g., a non-transitory computer-readable medium) is defined herein as a non-transitory memory device. A non-transitory memory device includes memory space located inside of a single physical storage device or memory space spread across multiple physical storage devices. When executed, software instructions stored in memory 154 may cause processor 146 to perform one or more processes described herein. Additionally or alternatively, hardwired circuitry may be used in place of or in combination with software instructions to perform one or more processes described herein. Thus, embodiments or aspects described herein are not limited to any specific combination of hardware circuitry and software.

RTC 156 may include a computer clock (e.g., in the form of an integrated circuit, etc.) that keeps track of the current time. For example, processor 146 may time stamp images and/or information associated with a medication delivery for a patient.

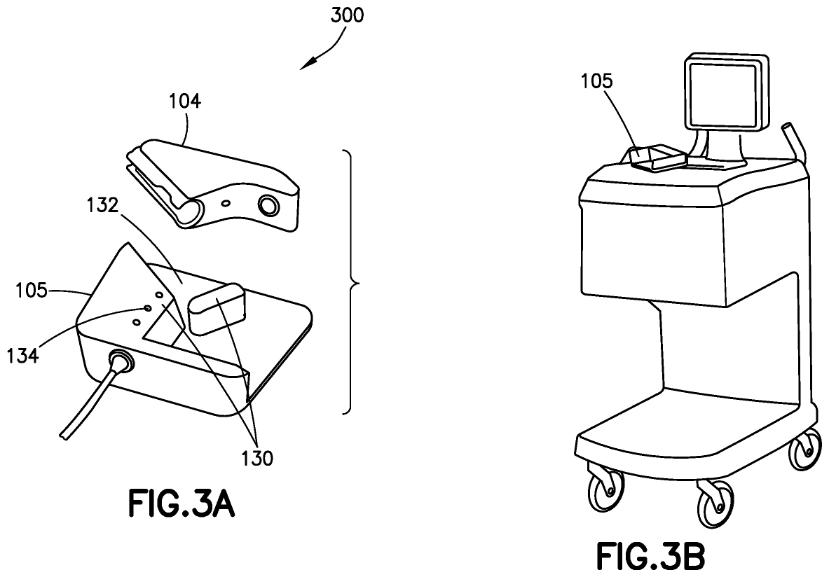

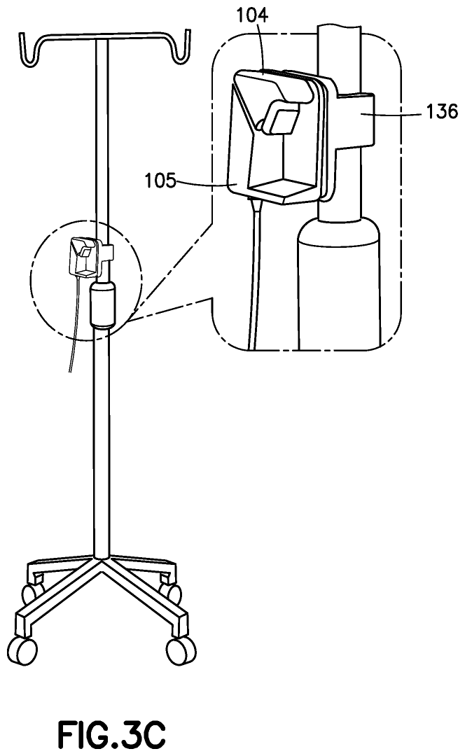

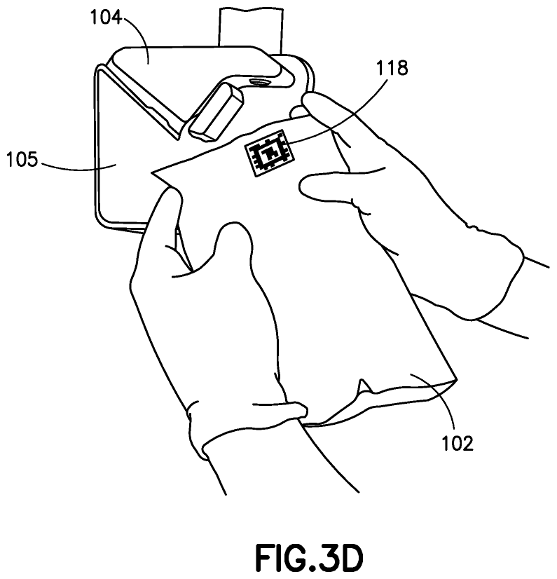

Base 105 may be configured to hold sensor assembly 104 and/or recharge a battery of power source 152 in sensor assembly 104 for reuse of sensor assembly 104. Further details regarding non-limiting embodiments or aspects of mechanical connector 142 are provided below with regard to FIGS. 3A-3D.

In some non-limiting embodiments or aspects, sensor assembly 104 may include a client device or a user device. For example, sensor assembly 104 may include one or more computing systems including one or more processors (e.g., one or more computing devices, one or more server computers, one or more mobile computing devices, one or more tablet computers, etc.). As an example, sensor assembly 104 may include a tablet computer or mobile computing device, such as an Apple® iPad, an Apple® iPhone, an Android® tablet, an Android® phone, and/or the like. For example, sensor assembly 104 may include a smartphone or a tablet carried by a nurse and/or another computing device (e.g., a nurse's workstation, a bedside station, etc.) located in a patient care area for managing patient care and/or EMR documentation. As an example, a display of such a computing device may function as user feedback device 150, such as to provide medication confirmation, administration information and/or guidance (e.g., delivery rate, contraindications, etc.), and/or dose administration rate feedback. In such an example, one or more of image capture device 144, processor 146, wireless communications circuitry 148, user feedback device 150, power source 152, memory 154, and/or real-time clock (RTC) 156 may be implemented by such a computing device, which need not include mechanical connector 142, and/or such a computing device may be in communication with one or more of image capture device 144, processor 146, wireless communications circuitry 148, user feedback device 150, power source 152, memory 154, and/or real-time clock (RTC) 156 in housing 140 connected to medical device 102 via mechanical connector 142. For example, such a computing device may be self-supported (e.g., via a phone or tablet stand, via a mobile nurse's workstation, etc.) at a location near the patient in a manner that enables the nurse to have a hand on medical device 102. In this way, if the user is already carrying a smartphone or a tablet to interact with the EMR, such a device may be used to implement sensor assembly 104 and/or one or more components of sensor assembly 104 (e.g., as an additional display or user feedback device, etc.).

Computing device 106 may include one or more devices capable of receiving information and/or data from sensor assembly 104 and/or one or more other computing devices and/or communicating information and/or data to sensor assembly 104 and/or the one or more other computing devices. For example, computing device 106 may include a server, a group of servers, a mobile device, a group of mobile devices, a receiving unit, a router device, a bridge device, a hub device, and/or the like. In some non-limiting embodiments or aspects, computing device 106 includes one or more computing devices, chips, contactless transmitters, contactless transceivers, NFC transmitters, RFID transmitters, Bluetooth® transceivers, contact based transmitters, and/or the like that enables computing device 106 to receive information directly from and/or communicate information directly to wireless communications circuitry 148 of sensor assembly 104 via a short range wireless communication connection (e.g., a communication connection that uses NFC protocol, a communication connection that uses Radio-frequency identification (RFID), a communication connection that uses a Bluetooth® wireless technology standard, and/or the like). In some non-limiting embodiments or aspects, computing device 106 may include and/or upload information and/or data to an electronic data management system, such as a hospital record system, a system used during a clinical trial to collect the trial related information, and/or the like.

The number and arrangement of devices and systems shown in FIGS. 1A and 1B is provided as an example. There may be additional devices and/or systems, fewer devices and/or systems, different devices and/or systems, or differently arranged devices and/or systems than those shown in FIGS. 1A and 1B. Furthermore, two or more devices and/or systems shown in FIGS. 1A and 1B may be implemented within a single device and/or system, or a single device and/or system shown in FIGS. 1A and 1B may be implemented as multiple, distributed devices and/or systems. Additionally, or alternatively, a set of devices and/or systems (e.g., one or more devices or systems) of environment 100 may perform one or more functions described as being performed by another set of devices and/or systems of environment 100.

Referring now to FIGS. 2A-2E, which illustrate an implementation 200 of non-limiting embodiments or aspects of sensor assembly 104, mechanical connector 142 may include opening 202 in a cylinder-shaped sidewall 204 extending from housing 140. For example, as shown in FIG. 2A, the sidewall of housing 140 may include may include a cylinder-shaped sidewall 204 extending along a length of housing 140 with opening 202 in the cylinder-shaped sidewall 204 extending in an axial direction of the cylinder shape 204 along the length of housing 140. Opening 202 may be configured to receive and/or hold lumen 102a and/or barrel 112 of syringe 102b.

Image capture device 144 may be located in a portion of the sidewall of housing 140 extending away from mechanical connector 142 in a direction perpendicular and/or at an angle to the length of the cylinder shape of mechanical connector 142 including opening 202. For example, a field-of-view of image capture device 144 may include a fixed field-of-view relative to housing 140 and/or mechanical connector 142 such that image capture device 144 may capture images of an area immediately proximate to and extending away from opening 202 at a first end of the cylinder shape closer to image capture device 144 than a second end of the cylinder shape opposite the first end (see, e.g., FIG. 2D). As an example, a focal plane of image capture device 144 may be at a fixed diagonal angle relative to housing 140 and/or mechanical connector 142. In such an example, the field-of-view of image capture device 144 may automatically include identifier elements 118 of lumen 102a and/or syringe 102b when lumen 102a and/or syringe 102b are connected to mechanical connector 142 (see, e.g., FIG. 2D).

As shown in FIGS. 2B and 2C, housing 140 may have a shape and size allowing a user to hold housing 140 in a single hand, for example, to scan or capture one or more images of identifier element 118 associated with a patient, and/or allowing a user to hold housing 140 in a first hand while connecting mechanical connector 142 to lumen 102a and/or a syringe 102b held in a second hand.

In some non-limiting embodiments or aspects, mechanical interface 142 including opening 202 may include a rigid deformable clip. For example, the cylinder shaped sidewall 204 of housing 140 surrounding opening 202 may be configured to clip onto an exterior surface of lumen 102a and/or barrel 112 of syringe 102b. As an example, the cylinder-shaped sidewall 204 may include a material forming the open cylinder shape that is configured to deform around and grasp an exterior surface of lumen 102a and/or barrel 112 of syringe 102b when mechanical interface 142 including opening 202 is pressed onto lumen 102a and/or barrel 112 of syringe 102b.

In some non-limiting embodiments or aspects, image capture device 144 may configured to automatically capture the one or more images in response to lumen 102a and/or barrel 112 of syringe 102b being received in opening 202. For example, the cylinder-shaped sidewall 204 of housing 140 within opening 202 may include a sensor (e.g., a mechanical sensor or button, a proximity sensor, an optical sensor, etc.) configured to detect a presence of lumen 102a and/or barrel 112 of syringe 102b within opening 202 and, in response thereto, provide a signal to processor 146 to control image capture device 144 to initiate capture of the one or more images.

Referring now to FIGS. 3A-3D, which illustrate an implementation 300 of non-limiting embodiments or aspects of base 105 for sensor assembly 104, base 105 may include a charging platform 130 having an opening 132 for receiving sensor assembly 104. The opening 132 may include charging pins which engage corresponding contacts in on the sidewall of housing 140 of sensor assembly 104 to charge a battery of power source 152 of sensor assembly 104 and/or charging platform may include wireless charging circuitry configured to wirelessly charge the battery of power source 152 of sensor assembly 104. Base 105 o charging platform 130, one or more LEDs may be configured to illuminate (e.g., a number of illuminated LEDs may indicate a level of charge of sensor assembly 104, a blinking LED may indicate charging of sensor assembly 104, etc.). As shown in FIG. 3B, base 105 may be configured to sit on a desk or nurse workstation. As shown in FIG. 3C, base 105 may include a clip 146 configured to attached base 105 to an IV pole. As shown in FIG. 3D, sensor assembly 104 may be configured to operate (e.g., capture the one or more images, process the one or more images, etc.) while located in base 105 and/or while charging. As an example, sensor assembly may be configured to automatically scan a medical device 102 (e.g., an IV medication bag, etc.) including identifier element 118 in response to a user placing the identifier element 118 within the field of view of image capture device 144 of sensor assembly 104.

Referring now to FIGS. 4A-4H, which illustrate an implementation 400 of non-limiting embodiments or aspects of sensor assembly 104, mechanical connector 142 may include a butterfly-style clip. For example, mechanical connector 142 may include jaws 402, each jaw 402 having a lower portion 404 connected together at a hinge (e.g., a spring, a pivot, a living hinge molded into a single one-piece clip device including housing 140, etc.) connected to housing 140. Each jaw 402 may include an inwardly facing surface configured to enclose around an exterior surface of medical device 102 (e.g., lumen 102a, barrel 112 of syringe 102b, an IV pole, etc.). The inwardly facing surface of each jaw 402 may include an outward bulge or curved recess 405 at a portion of the inwardly facing surface of jaws 402 configured to contact the exterior surface of medical device 102 (e.g., lumen 102a, barrel 112 of syringe 102b, an IV pole, etc.) when the jaws 402 are enclosed around the exterior surface of medical device 102. The hinge may be configured to urge the lower portions 404 of the jaws 402 together such that the inwardly facing surface of the jaws 402 secure medical device 102 between the jaws 402 (e.g., at the bulge 405, etc.). Each of the jaws 402 may have a handle 406 extending from an upper portion opposite the lower portion 404 thereof, which when moved towards each other, against a force of the hinge, causes the lower portions 404 to separate from each other (see, e.g., FIG. 4C), for example, to be attached to lumen 102a (see, e.g., FIG. 4D).

In a same or similar manner to implementation 300, image capture device 144 may be located in a portion of the sidewall of housing 140 extending away from mechanical connector 142 in a direction perpendicular and/or at an angle to a space between jaws 402. For example, a field-of-view of image capture device 144 may include a fixed field-of-view relative to housing 140 and/or mechanical connector 142 such that image capture device 144 may capture images of an area immediately proximate to and extending away from a first side of jaws 402 closer to image capture device 144 than second side of jaws 402 opposite the first side (see, e.g., FIG. 4D). In such an example, the field-of-view of image capture device 144 may automatically include identifier elements 118 of lumen 102a and/or syringe 102b when lumen 102a and/or syringe 102b are connected to mechanical connector 142 (see, e.g., FIGS. 4F and 4G).

Again in a same or similar manner to implementation 300, and as shown in FIGS. 4B and 4C, housing 140 may have a shape and size allowing a user to hold housing 140 in a single hand, for example, to scan or capture one or more images of identifier element 118 associated with a patient, and/or allowing a user to hold housing 140 in a first hand while connecting mechanical connector 142 to lumen 102a and/or a syringe 102b held in a second hand.

In some non-limiting embodiments or aspects, image capture device 144 may configured to automatically capture the one or more images in response actuation of handles 406 (e.g., in response to actuation of the butterfly-style clip, etc.). For example, housing 140 (e.g., a the hinge between jaws 402, etc.) may include a sensor (e.g., a mechanical sensor or button, a proximity sensor, an optical sensor, etc.) configured to detect an actuation of the jaws 402 and/or a presence of lumen 102a and/or barrel 112 of syringe 102b between the jaws 402 and, in response thereto, provide a signal to processor 146 to control image capture device 144 to initiate capture of the one or more images.

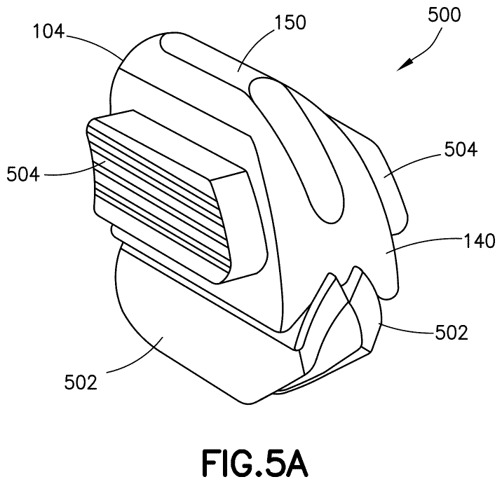

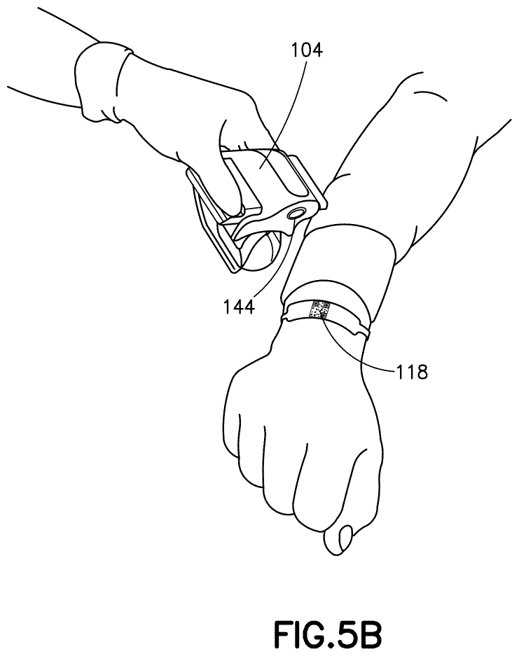

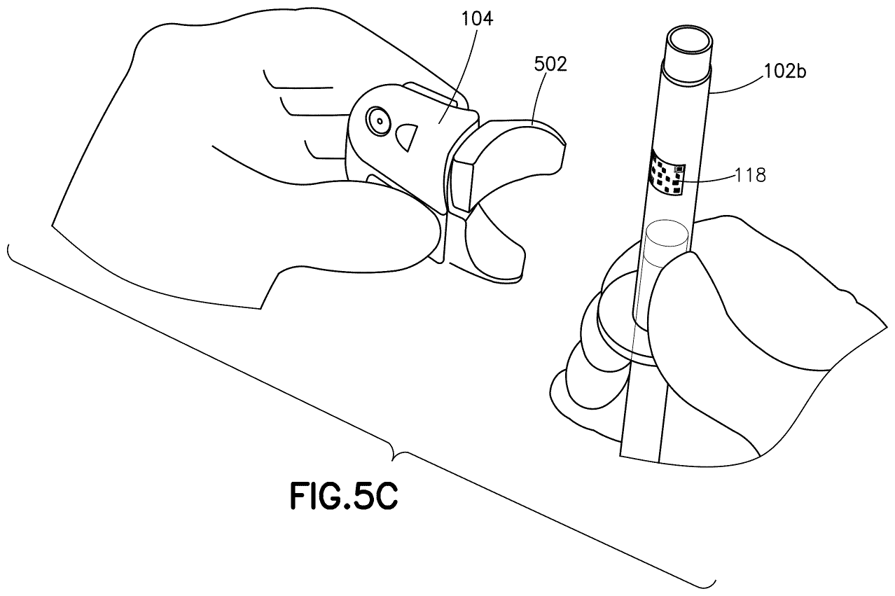

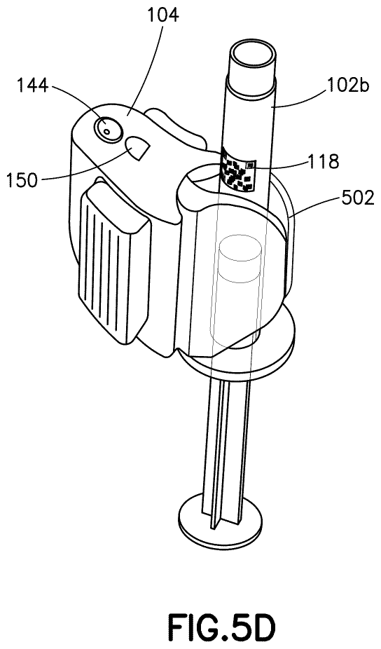

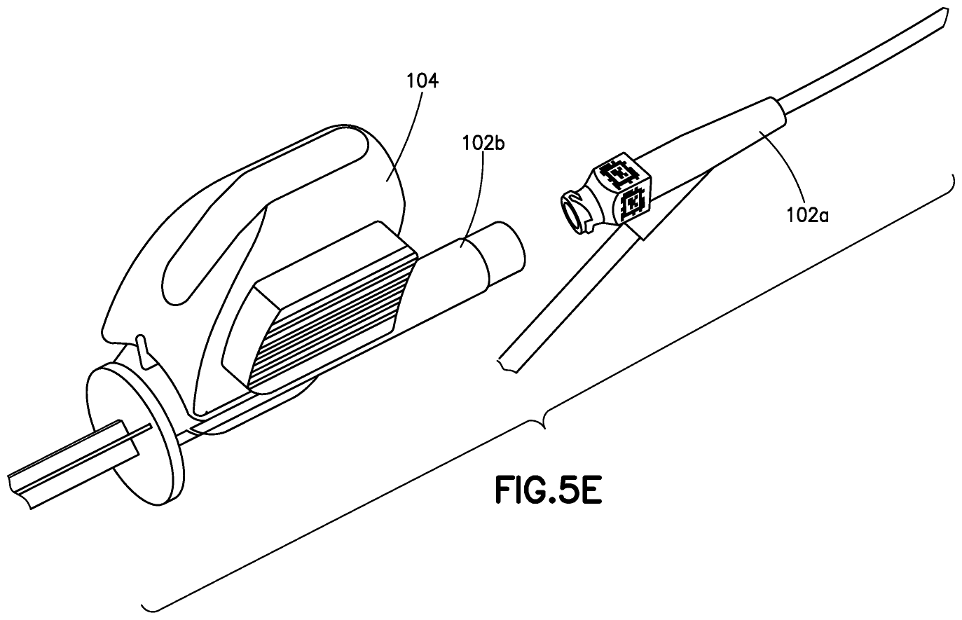

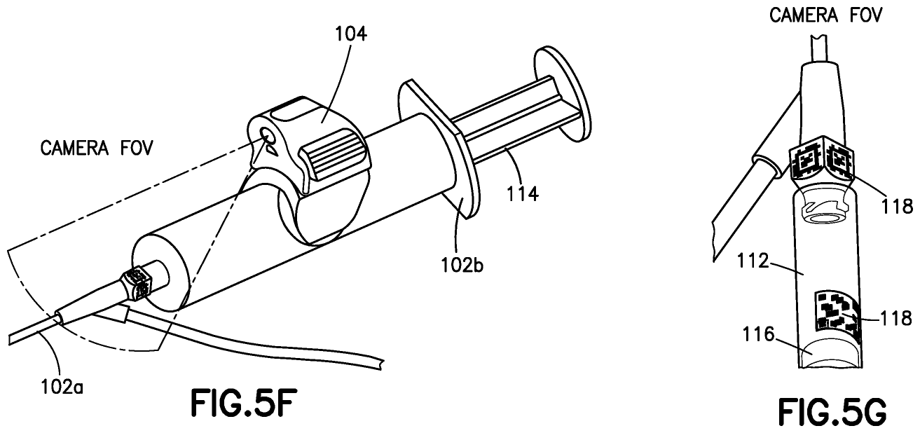

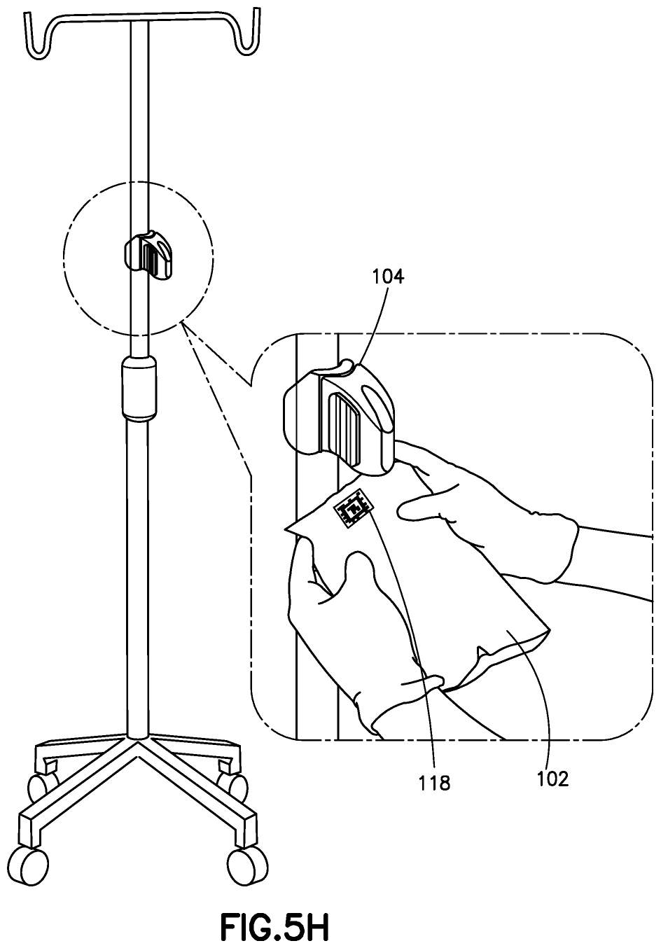

Referring now to FIGS. 5A-5H, which illustrate an implementation 500 of non-limiting embodiments or aspects of sensor assembly 104, mechanical connector 142 may include a button-actuated clip. For example, mechanical connector 142 may include arcuate jaws 502 extending from housing 140, each arcuate jaw 502 connected together at a first end to a hinge (e.g., a spring, a pivot, a living hinge molded into a single one-piece clip device including housing 140, etc.) within housing 140. Each arcuate jaw 502 may include an inwardly facing surface configured to enclose around an exterior surface of medical device 102 (e.g., lumen 102a, barrel 112 of syringe 102b, an IV pole, etc.). The hinge may be configured to urge second ends of the arcuate jaws 502 opposite the first ends together such that the inwardly facing surface of arcuate jaws 502 secure medical device 102 between arcuate jaws 502. Mechanical connector 142 may include one or more buttons 504 extending from the sidewall of housing 140. Actuation of the one or more buttons 504 may cause the arcuate jaws 502 to separate from each other (see, e.g., FIG. 5C), for example, to be attached to syringe 102b (see, e.g., FIG. 4D).

In a same or similar manner to implementations 300 and/or 400, image capture device 144 may be located in a portion of the sidewall of housing 140 extending away from mechanical connector 142 in a direction perpendicular and/or at an angle to a space between arcuate jaws 502. For example, a field-of-view of image capture device 144 may include a fixed field-of-view relative to housing 140 and/or mechanical connector 142 such that image capture device 144 may capture images of an area immediately proximate to and extending away from a first side of arcuate jaws 502 closer to image capture device 144 than a second side of arcuate jaws 502 opposite the first side (see, e.g., FIGS. 5F and 5G). In such an example, the field-of-view of image capture device 144 may automatically include identifier elements 118 of lumen 102a and/or syringe 102b when lumen 102a and/or syringe 102b are connected to mechanical connector 142 (see, e.g., FIGS. 5F and 5G).

Again in a same or similar manner to implementations 300 and/or 400, and as shown in FIGS. 5B and 5C, housing 140 may have a shape and size allowing a user to hold housing 140 in a single hand, for example, to scan or capture one or more images of identifier element 118 associated with a patient, and/or allowing a user to hold housing 140 in a first hand while connecting mechanical connector 142 to lumen 102a and/or a syringe 102b held in a second hand.

In some non-limiting embodiments or aspects, image capture device 144 may configured to automatically capture the one or more images in response actuation of the one or more buttons 504. For example, housing 140 may include a sensor (e.g., a mechanical sensor or button, a proximity sensor, an optical sensor, etc.) configured to detect an actuation of the one or more buttons and/or a presence of lumen 102a and/or barrel 112 of syringe 102b between the jaws 402 and, in response thereto, provide a signal to processor 146 to control image capture device 144 to initiate capture of the one or more images.

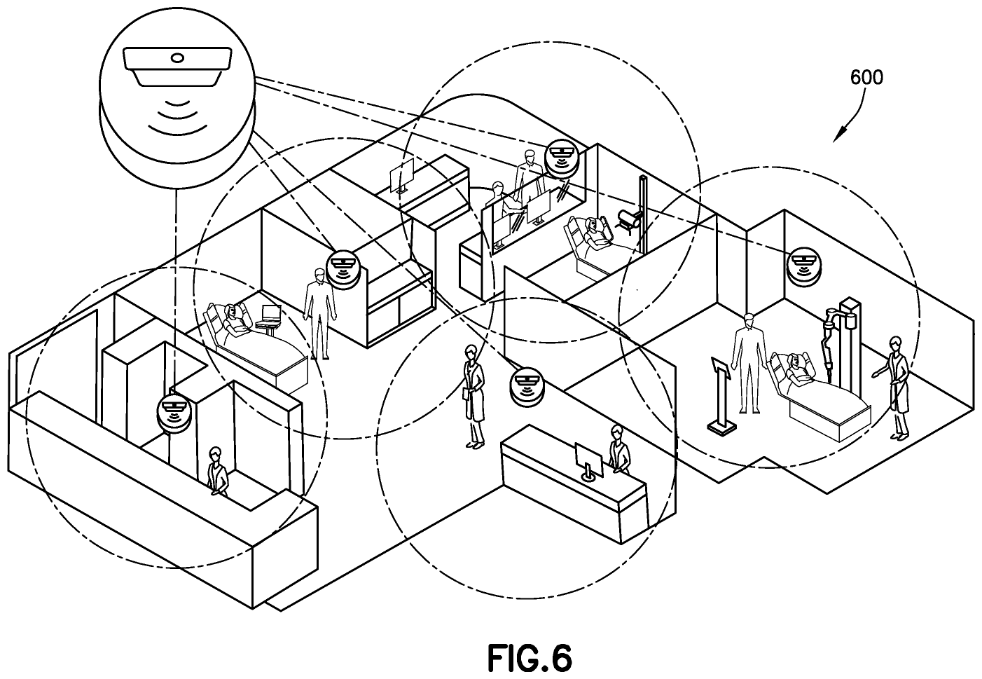

Referring now to FIG. 6, FIG. 6 illustrates an implementation 600 of non-limiting embodiments or aspects of a communication network for a sensor assembly. As shown in FIG. 6, the communication network may include a plurality of hubs 602 (e.g., Bluetooth® hubs, etc.) located throughout a hospital to create communication cells at locations where Internet of Medical Things (IoMT) devices, such as sensor assembly 104, and/or the like are expected to be used and/or at locations where tracking is desired. For example, low power requirements of Bluetooth® radios may enable small low-cost consumable devices or sensors (e.g., small batteries, small form factors, etc.) and/or support data upload from an ecosystem of such small devices or sensors. Seamless and secure roaming from or hub-to-hub may be managed by a central IoT access controller, which may be implemented by external computing device 106, and/or the like, and which may use triangulation to accurately determine a location each device or sensor (e.g., each sensor assembly 104, etc.).

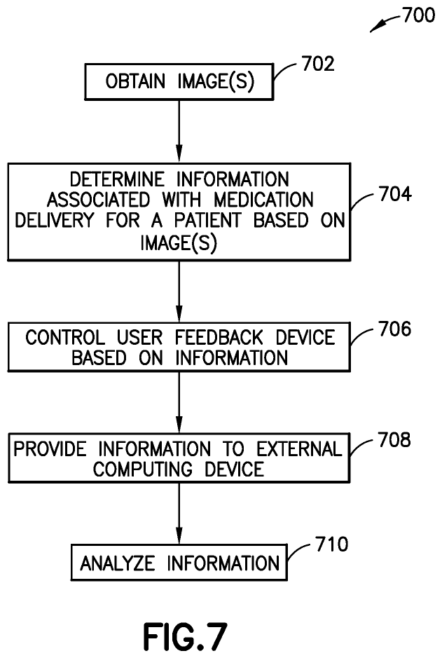

Referring now to FIG. 7, FIG. 7 is a flowchart of non-limiting embodiments or aspects of a process 700 for monitoring push medication delivery to patients from syringes. In some non-limiting embodiments or aspects, one or more of the steps of process 700 may be performed (e.g., completely, partially, etc.) by sensor assembly 104 (e.g., one or more devices of sensory assembly 104, etc.). In some non-limiting embodiments or aspects, one or more of the steps of process 700 may be performed (e.g., completely, partially, etc.) by another device or a group of devices separate from or including sensor assembly 104, such as computing device 106 (e.g., one or more devices of a system of computing device 106, etc.).

As shown in FIG. 7, at step 702, process 700 includes obtaining one or more images captured by an image capture device. For example, processor 146 of sensor assembly 104 (and/or computing device 106) may obtain one or more images captured by image capture device 144. As an example, image capture device 144 of sensor assembly 104 may capture the one or more images (e.g., a plurality of images captured over a period of time, etc.). In such an example, image capture device 144 may capture the one or more images in response to a connection of mechanical connector 142 to lumen 102a and/or syringe 102b and/or an in response to an actuation of mechanical connector 142.

As shown in FIG. 7, at step 704, process 700 includes determining, based on the one or more images, information associated with a medication delivery for a patient. For example, processor 146 of sensor assembly 104 (and/or computing device 106) may determine, based on the one or more images, information associated with a medication delivery for a patient. As an example, information associated with a medication delivery for a patient may include, but are not limited to, 1) information about a medication source such as type of medication, a volume of medication, a concentration of medication, etc.; 2) constant patient-specific information such as patient identification number, drug allergies, blood type, etc.; 3) variable patient-specific information such as patient vital signs, lab results, current disease states and/or clinical diagnoses, drugs previously administered, etc.; 4) medical orders such as drug, dose, route of administration, treatment schedule, etc.; 5) clinical guidelines such as known drug-drug interactions, recommended treatment protocols, dosing limits, etc.; 6) environmental factors such as the care area where treatment is being delivered, time of day, date, temperature, etc.; 7) valve status such as currently open (second state), currently closed (first state) or clinician initiation of a manual override; 8) historic patient information such as disease state, clinical diagnosis, dosing history, etc.; and 9) any other relevant information applicable to determining whether or a not a particular fluid administration is safe and appropriate for a patient. For example, information associated with a medication delivery for a patient may include patient information, lumen information, syringe information, flush syringe information, and/or the like.

Patient information may include at least one of the following parameters associated with a patient: a unique patient identifier, an approved medication, an approved medication dosage, an approved medication delivery lumen, an approved medication delivery time, a patient medication allergy, or any combination thereof.

Lumen information may include at least one of the following parameters associated with a lumen: a unique lumen identifier, a unique patient identifier of a patient associated with the lumen; identifiers of one or more other medical devices connected to and/or forming the lumen, one or more previous medication previously delivered via the lumen, or any combination thereof.

Syringe information may include at least one of the following parameters associated with a syringe: a unique syringe identifier, a type of medication included in a syringe, a dosage of the medication included in the syringe, a concentration of the medication included in the syringe, a prescribed rate of delivery of the medication included in the syringe, or any combination thereof.

Flush syringe information may include at least one of the following parameters associated with a flush syringe: a unique flush syringe identifier, a type of flush fluid contained in the flush syringe, a volume of flush fluid contained in the flush syringe, or any combination thereof.

In some non-limiting embodiments or aspects, processor 146 of sensor assembly 104 (and/or computing device 106) may determine, based on the one or more images, a patient identifier, a lumen identifier, a syringe identifier, and/or a flush syringe identifier and, access, using the patient identifier, the lumen identifier, the syringe identifier, and/or the flush syringe identifier a database (e.g., at computing device 106, etc.) and/or a look-up table to determine patient information, lumen information, syringe information, flush syringe information associated therewith.

In some non-limiting embodiments or aspects, information associated with a medication delivery for a patient may include a caregiver (e.g., nurse, etc.) identifier associated with sensor assembly 104 and/or a room identifier associated with sensor assembly 104. For example, sensor assembly may be assigned to a particular caregiver and/or to a particular room of a hospital. As an example, the caregiver identifier and/or the room identifier may be used (e.g., by computing device 106, etc.) to calculate one or more analytics associated with the particular caregiver and/or the particular room.

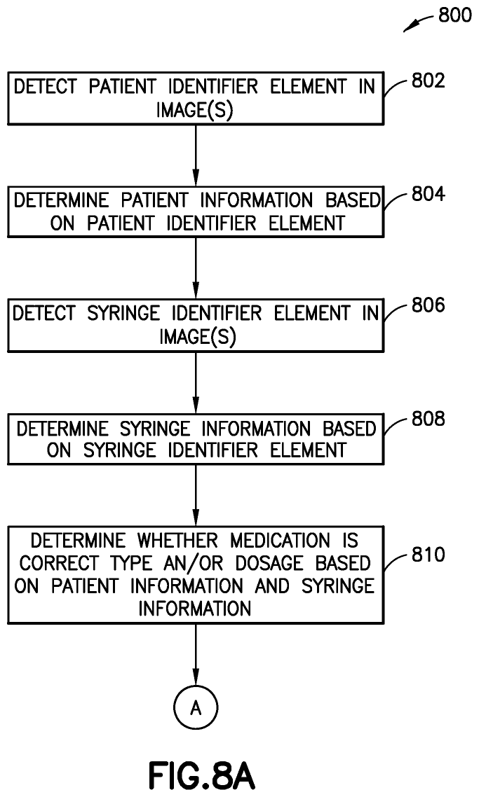

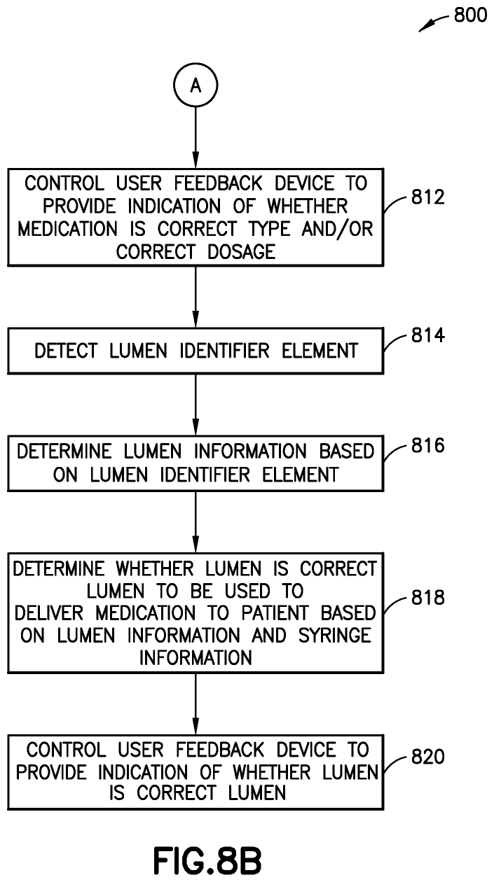

Further details regarding non-limiting embodiments or aspects of step 704 of process 700 are provided below with regard to FIGS. 8A, 8B, 9, and 10.

As shown in FIG. 7, at step 706, process 700 includes controlling a user feedback device based on the information associated with the medication delivery for the patient. For example, processor 146 of sensor assembly 104 (and/or computing device 106) may control, based on the information associated with the medication delivery for the patient, user feedback device 150 to provide the audible and/or visual output to the user.

Further details regarding non-limiting embodiments or aspects of step 706 of process 700 are provided below with regard to FIGS. 8A, 8B, 9, and 10.

As shown in FIG. 7, at step 708, process 700 includes providing the information associated with the medication delivery for the patient to an external computing device. For example, processor 146 of sensor assembly 104 may control wireless communications circuitry 148 to communicate the information associated with the medication delivery for the patient to computing device 106. As an example, processor 146 of sensor assembly 104 may control wireless communications circuitry 148 to communicate one or more images of one or more medical devices 102 (e.g., syringe 102b, etc.) connected to lumen 102a to computing device 106.

As shown in FIG. 7, at step 710, process 700 includes analyzing the information associated with the medication delivery for the patient. For example, computing device 106 may analyze the information associated with the medication delivery for the patient. As an example, computing device 106 may review the one or more images to verify that one or more medical devices 102 (e.g., syringe 102b, etc.) were validly connected to lumen 102a. As an example, computing device 106 may compute and/or display analytics based on the information associated with the medication delivery for the patient received from a plurality of sensor assemblies 104 and/or associated with a plurality of patients, which may enable detecting patterns of risky behavior and/or the like.

Referring now to FIGS. 8A and 8B, FIGS. 8A and 8B are a flowchart of non-limiting embodiments or aspects of a process 800 for monitoring push medication delivery to patients from syringes. In some non-limiting embodiments or aspects, one or more of the steps of process 800 may be performed (e.g., completely, partially, etc.) by sensor assembly 104 (e.g., one or more devices of sensory assembly 104, etc.). In some non-limiting embodiments or aspects, one or more of the steps of process 700 may be performed (e.g., completely, partially, etc.) by another device or a group of devices separate from or including sensor assembly 104, such as computing device 106 (e.g., one or more devices of a system of computing device 106, etc.).

As shown in FIG. 8A, at step 802, process 800 includes detecting, in the one or more images, a patient identifier element 118 associated with the patient. For example, processor 146 of sensor assembly 104 (and/or computing device 106) may detect, in the one or more images, a patient identifier element 118 associated with the patient.

As shown in FIG. 8A, at step 804, process 800 includes determining, based on the detected patient identifier element 118, patient information associated with the patient. For example, processor 146 of sensor assembly 104 (and/or computing device 106) may determine, based on the detected patient identifier element 118, patient information associated with the patient. As an example, the patient information may include an indication of a type and/or a dosage of a medication to be delivered to the patient and/or a unique identifier of a medication delivery lumen to be used to deliver the medication to the patient.

As shown in FIG. 8A, at step 806, process 800 includes detecting, in the one or more images, a syringe identifier element 118 associated with the syringe. For example, processor 146 of sensor assembly 104 (and/or computing device 106) may detect, in the one or more images, a syringe identifier element 118 associated with syringe 102b.

As shown in FIG. 8A, at step 808, process 800 includes determining, based on the detected syringe identifier element 118, syringe information associated with the syringe. For example, processor 146 of sensor assembly 104 (and/or computing device 106) may determine, based on the detected syringe identifier element 118, syringe information associated with syringe 102b. As an example, the syringe information may include a type and/or a dosage of a medication contained in syringe 102b.

As shown in FIG. 8A, at step 810, process 800 includes determining, based on the patient information and the syringe information, whether the medication contained in the syringe is a correct type and/or a correct dosage of the medication to be delivered to the patient. For example, processor 146 of sensor assembly 104 (and/or computing device 106) may determine, based on the patient information and the syringe information, whether the medication contained in syringe 102b is a correct type and/or a correct dosage of the medication to be delivered to the patient. As an example, processor 146 of sensor assembly 104 (and/or computing device 106) may compare medication, medication dosage, medication delivery route or IV line, and/or medication delivery time associated with an IV line to an approved patient, approved medication, an approved medication dosage, an approved medication delivery lumen, an approved medication delivery time associated with the patient identifier and/or a patient medication allergy to reduce medication administration errors.

As shown in FIG. 8B, at step 812, process 800 includes controlling, based on whether the medication contained in the syringe includes the correct type and/or the correct dosage of the medication to be delivered to the patient, the user feedback device to provide the audible and/or visual output to the user. For example, processor 146 of sensor assembly 104 (and/or computing device 106) may control, based on whether the medication contained in syringe 102b includes the correct type and/or the correct dosage of the medication to be delivered to the patient, user feedback device 150 to provide the audible and/or visual output to the user. As an example, if it is determined that the medication contained in syringe 102b does not include the correct type and/or the correct dosage of the medication to be delivered to the patient, processor 146 of sensor assembly 104 (and/or computing device 106) may control user feedback device 150 to issue an alert (e.g., red flashing LEDs, etc.) indicating that the medication contained in syringe 102b is an improper medication for the patient, an improper dosage for the patient and/or medication, an improper medication delivery route for the patient and/or medication (e.g., improper lumen 102a), an improper medication delivery time for the patient and/or medication, and/or a patient medication allergy. As an example, if it is determined that the medication contained in syringe 102b includes the correct type and/or the correct dosage of the medication to be delivered to the patient, processor 146 of sensor assembly 104 (and/or computing device 106) may control user feedback device 150 to issue an alert (e.g., green flashing LEDs, etc.) indicating that the medication contained in syringe 102b is approved for delivery to the patient.

As shown in FIG. 8B, at step 814, process 800 includes detecting, in the one or more images, a lumen identifier element 118 associated with the lumen. For example, processor 146 of sensor assembly 104 (and/or computing device 106) may detect, in the one or more images, a lumen identifier element 118 associated with lumen 102a.

As shown in FIG. 8B, at step 816, process 800 includes determining, based on the detected lumen identifier element 118, lumen information associated with the lumen. For example, processor 146 of sensor assembly 104 (and/or computing device 106) may determine, based on the detected lumen identifier element 118, lumen information associated with lumen 102a. As an example, the lumen information may include a unique identifier of lumen 102a.

As shown in FIG. 8B, at step 818, process 800 includes determining, based on the lumen information and the syringe information, whether the lumen is the medication delivery lumen to be used to deliver the medication to the patient. For example, processor 146 of sensor assembly 104 (and/or computing device 106) may determine, based on the lumen information and the syringe information, whether lumen 102a is the medication delivery lumen (e.g., a correct lumen of a plurality of lumens associated with the patient, etc.) to be used to deliver the medication to the patient. As an example, processor 146 of sensor assembly 104 (and/or computing device 106) may compare the identifier of lumen 102a to an approved medication delivery lumen.

As shown in FIG. 8B, at step 820, process 800 includes controlling, based on whether the lumen includes the medication delivery lumen to be used to deliver the medication to the patient, the user feedback device to provide the audible and/or visual output to the user. For example, processor 146 of sensor assembly 104 (and/or computing device 106) may control, based on whether lumen 102a includes the medication delivery lumen to be used to deliver the medication to the patient, user feedback device 150 to provide the audible and/or visual output to the user. As an example, if it is determined that lumen 102a does not include the medication delivery lumen to be used to deliver the medication to the patient, processor 146 of sensor assembly 104 (and/or computing device 106) may control user feedback device 150 to issue an alert (e.g., red flashing LEDs, etc.) indicating that lumen 102a is an improper lumen for the medication delivery. As an example, if it is determined that that lumen 102a includes the medication delivery lumen to be used to deliver the medication to the patient, processor 146 of sensor assembly 104 (and/or computing device 106) may control user feedback device 150 to issue an alert (e.g., green flashing LEDs, etc.) indicating that lumen 102a is approved for the medication delivery.

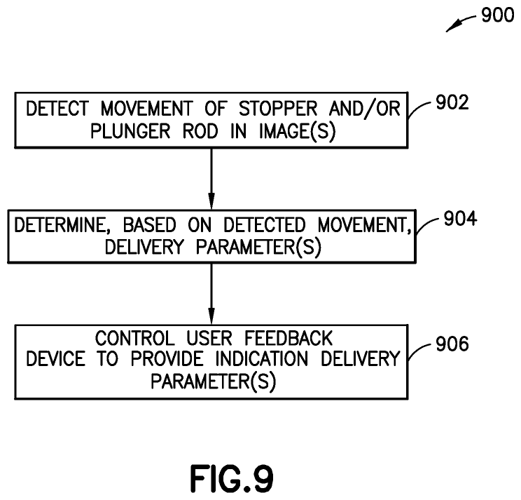

Referring now to FIG. 9, FIG. 9 is a flowchart of non-limiting embodiments or aspects of a process 900 for monitoring push medication delivery to patients from syringes. In some non-limiting embodiments or aspects, one or more of the steps of process 900 may be performed (e.g., completely, partially, etc.) by sensor assembly 104 (e.g., one or more devices of sensory assembly 104, etc.). In some non-limiting embodiments or aspects, one or more of the steps of process 900 may be performed (e.g., completely, partially, etc.) by another device or a group of devices separate from or including sensor assembly 104, such as computing device 106 (e.g., one or more devices of a system of computing device 106, etc.).

As shown in FIG. 9, at step 902, process 900 includes detecting, in the one or more images, a movement of a stopper and/or a plunger rod of the syringe. For example, processor 146 of sensor assembly 104 (and/or computing device 106) may detect, in the one or more images, a movement of stopper 116 and/or plunger rod 114 of syringe 102b. As an example, the one or more images may include a series of images including syringe 102b during the medication delivery for the patient. As an example, processor 146 of sensor assembly 104 (and/or computing device 106) may monitor medication fluid delivery and/or lumen flushing by tracking movement of an actuation mechanism or fluid expulsion mechanism, such as stopper 116 and/or plunger rod 114 of syringe 102b, during the fluid delivery procedure and/or the flushing procedure.

As shown in FIG. 9, at step 904, process 900 includes determining, based on the detected movement and/or the syringe information, at least one delivery parameter of the following delivery parameters: a patency of the lumen (e.g., a catheter patency, etc.); an average rate of delivery of the medication; a current rate of the delivery of the medication; a steadiness index of the current rate of delivery of the medication; a volume of the medication delivered; a completion of the medication delivery for the patient; or any combination thereof. For example, processor 146 of sensor assembly 104 (and/or computing device 106) may determine, based on the detected movement, at least one of the following: a patency of the lumen (e.g., a catheter patency, etc.); an average rate of delivery of the medication, a current rate of the delivery of the medication; a steadiness index of the current rate of delivery of the medication, a volume of the medication delivered; a completion of the medication delivery for the patient; or any combination thereof. In such an example, a steadiness index of the current rate of delivery of the medication may include an indication of whether the current rate of delivery of the medication matches, is less than, or is greater than a prescribed or ideal rate of delivery of the medication. In such an example, a patency of the lumen may include an indication (e.g., a yes or positive, a no or negative, a percentage, etc.) of the patency of the lumen.

In some non-limiting embodiments or aspects, identifier element 118 of syringe 102b may be used by processor 146 of sensor assembly 104 (and/or computing device 106) to estimate a position of stopper 116 and/or plunger rod 114 of syringe 102b. For example, image processing software may record an initial position of stopper 116 and/or plunger rod 114 of syringe 102b relative to the position of identifier element 118 and, when stopper 116 and/or plunger rod 114 of syringe 102b moves relative to the position of identifier element 118, the image processing software may determine that a medication delivery procedure has begun. When stopper 116 and/or plunger rod 114 of syringe 102b advances a predetermined distance from identifier element 118, it may be assumed that the fluid delivery procedure is complete.

In some non-limiting embodiments or aspects, processor 146 of sensor assembly 104 (and/or computing device 106) may automatically identify the position of stopper 116 and/or plunger rod 114 of syringe 102b relative to other markers on syringe 102b. For example, the markings may be graduated lines and/or indicia on syringe barrel 112. As an example, the movement of stopper 116 and/or plunger rod 114 of syringe 102b relative to the markings may be used determine not only initiation and dose, but also fluid volume delivered.

In some non-limiting embodiments or aspects, processor 146 of sensor assembly 104 (and/or remote computing device 106) may determine, in response to detecting, in the one or more images, a backward or proximal movement of the stopper and/or the plunger rod of the syringe that satisfies a threshold distance, a positive lumen patency. For example, a flush syringe may be used to check catheter patency by aspirating (e.g., pulling back, etc.) on the plunger of the syringe, and if processor 146 of sensor assembly 104 (and/or remote computing device 106) determines that the plunger of the syringe has moved in a backward direction a distance that satisfies the threshold distance, processor 146 of sensor assembly 104 (and/or remote computing device 106) may confirm the positive catheter patency and/or provide an indication of the positive catheter patency to the user (E.g., via user feedback device 150, etc.). In some non-limiting embodiments or aspects, processor 146 of sensor assembly 104 (and/or remote computing device 106) may additionally, or alternatively, confirm the positive lumen patency in response to detecting, in the one or more images (e.g., using the image processing software, etc.), blood in the lumen (e.g., in the catheter line, etc.).

As shown in FIG. 9, at step 906, process 900 includes controlling, based on the at least one delivery parameter, the user feedback device to provide the audible and/or visual output to the user. For example, processor 146 of sensor assembly 104 (and/or computing device 106) may control, based on the at least one delivery parameter, user feedback device 150 to provide the audible and/or visual output to the user. As an example, the audible and/or visual output to the user may be associated with the at least one delivery parameter.

In some non-limiting embodiments or aspects, processor 146 of sensor assembly 104 (and/or computing device 106) may control user feedback device 150 to provide guidance on rate of administration or delivery of the medication (e.g., fast, slow, “over X minutes”, etc.). As an example, processor 146 of sensor assembly 104 (and/or computing device 106) may compare, during the fluid delivery procedure a current delivery rate to a prescribed or approved delivery rate for the medication and, based on the comparison, provide guidance to maintain the delivery rate, increase the delivery rate, and/or reduce the delivery rate. For example, during medication delivery, the current rate of delivery of the medication may be variable over a delivery time period, and processor 146 of sensor assembly 104 (and/or computing device 106) may compare the current rate of delivery to the prescribed rate or an ideal steady rate and determine whether the current rate of delivery should be increased, decreased, or maintained to match the prescribed or ideal rate of delivery (and/or control user feedback device 150 to provide a prompt to the user to increase, decrease, or maintain the current rate of delivery to match the prescribed or ideal rate of delivery). As an example, some users may attempt to maintain a steady rate of delivery over a delivery time period (e.g., 1 minute, etc.); however, other users may deliver ¼^{th} a dose rapidly 4 separate times over the delivery time period, which may be a less ideal delivery from a pharmacokinetics perspective than the more steady rate of delivery over the delivery time period. In this way, non-limiting embodiment or aspects of the present disclosure may improve a steadiness of medication delivery.

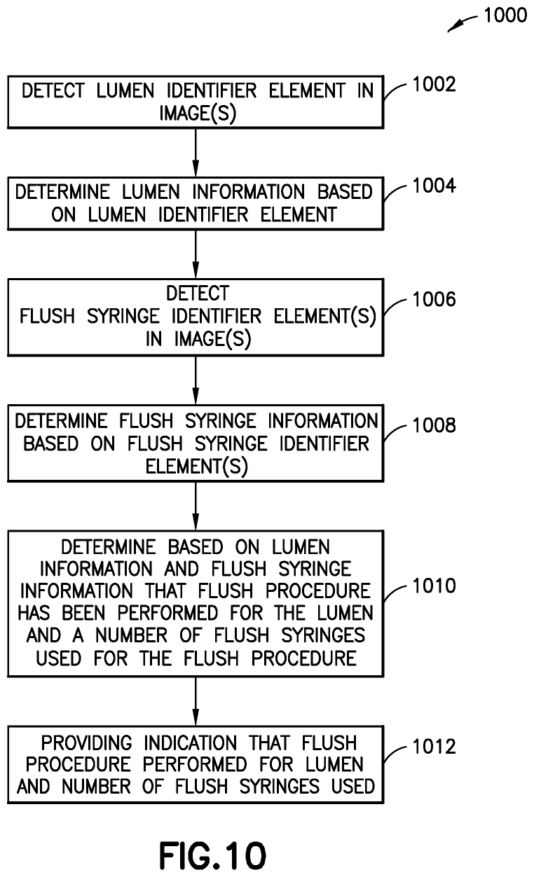

Referring now to FIG. 10, FIG. 10 is a flowchart of non-limiting embodiments or aspects of a process 1000 for monitoring push medication delivery to patients from syringes. In some non-limiting embodiments or aspects, one or more of the steps of process 1000 may be performed (e.g., completely, partially, etc.) by sensor assembly 104 (e.g., one or more devices of sensory assembly 104, etc.). In some non-limiting embodiments or aspects, one or more of the steps of process 1000 may be performed (e.g., completely, partially, etc.) by another device or a group of devices separate from or including sensor assembly 104, such as computing device 106 (e.g., one or more devices of a system of computing device 106, etc.).

As shown in FIG. 10, at step 1002, process 1000 includes detecting, in the one or more images, a lumen identifier element 118 associated with the lumen. For example, processor 146 of sensor assembly 104 (and/or computing device 106) may detect, in the one or more images, a lumen identifier element 118 associated with lumen 102a.

As shown in FIG. 10, at step 1004, process 1000 includes determining, based on the detected lumen identifier element 118, lumen information associated with the lumen. For example, processor 146 of sensor assembly 104 (and/or computing device 106) may determine, based on the detected lumen identifier element 118, lumen information associated with lumen 102a. As an example, the lumen information may include a unique identifier of lumen 102a.

As shown in FIG. 10, at step 1006, process 1000 includes detecting, in the one or more images, one or more flush syringe identifier elements 118 associated with the one or more flush syringes. For example, processor 146 of sensor assembly 104 (and/or computing device 106) may detect, in the one or more images, one or more flush syringe identifier elements 118 associated with the one or more flush syringes. As an example, the one or more images may include a series of images including one or more flush syringes connected to lumen 102a during a flush procedure.

As shown in FIG. 10, at step 1008, process 1000 includes determining, based on the one or more detected flush syringe identifier elements 118, flush syringe information associated with the one or more flush syringes. For example, processor 146 of sensor assembly 104 (and/or computing device 106) may determine, based on the one or more detected flush syringe identifier elements 118, flush syringe information associated with the one or more flush syringes. As an example, the flush syringe information may include a number of flush syringes used for the flush procedure (e.g., a number of flush syringes connected to lumen 102a during the flush procedure, etc.).

As shown in FIG. 10, at step 1001, process 1000 includes determining, based on the lumen information and the flush syringe information, that the flush procedure has been performed for the lumen, a number of flush syringes used for the flush procedure, and/or a type of the flush procedure performed (e.g., continuous flushing, partial flushing, pulsatile flushing, etc.). For example, processor 146 of sensor assembly 104 (and/or computing device 106) may determine, based on the lumen information and the flush syringe information, that the flush procedure has been performed for lumen 102a, a number of flush syringes used for the flush procedure, and/or a type of the flush procedure performed (e.g., continuous flushing, partial flushing, pulsatile flushing, etc.).

As shown in FIG. 10, at step 1012, process 1000 includes providing an indication that that the flush procedure has been performed for the lumen and the number of flush syringes used for the flush procedure. For example, processor 146 of sensor assembly 104 (and/or computing device 106) may provide an indication that that the flush procedure has been performed for lumen 102a and the number of flush syringes used for the flush procedure. As an example, processor 146 of sensor assembly 104 may control wireless communications circuitry 148 to communicate the indication to computing device 106.

Although embodiments or aspects have been described in detail for the purpose of illustration and description, it is to be understood that such detail is solely for that purpose and that embodiments or aspects are not limited to the disclosed embodiments or aspects, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, any of these features can be combined in ways not specifically recited in the claims and/or disclosed in the specification. Although each dependent claim listed below may directly depend on only one claim, the disclosure of possible implementations includes each dependent claim in combination with every other claim in the claim set.

What is claimed is:

1. A sensor assembly, comprising:
an image capture device configured to capture one or more images;
a user feedback device configured to provide an audible and/or visual output to a user;
a mechanical connector configured to connect the sensor assembly to a lumen and/or a syringe configured to be connected to the lumen; and
a processor coupled to a memory and configured to:
obtain the one or more the images captured by the image capture device;
determine, based on the one or more images, information associated with a medication delivery for a patient; and
control, based on the information, the user feedback device to provide the audible and/or visual output to the user.

2. The sensor assembly of claim 1, wherein the image capture device is configured to automatically capture the one or more images in response to a connection of the mechanical connector to the lumen and/or the syringe.

3. The sensor assembly of claim 1, further comprising:
a housing including the image capture device, the user feedback device, and the processor, wherein the mechanical connector extends from a sidewall of the housing, wherein a field-of-view of the image capture device is fixed relative to the housing and/or the mechanical connector, and wherein, with the mechanical connector connecting the sensor assembly to the lumen and/or the syringe connected to the lumen, a field-of-view of the image capture device includes the syringe.

4. The sensor assembly of claim 3, wherein the mechanical connector includes an opening in a sidewall extending from the housing, and wherein the opening is configured to receive the lumen and/or a barrel of the syringe.

5. The sensor assembly of claim 4, wherein the image capture device is configured to automatically capture the one or more images in response to the lumen and/or the barrel of the syringe being received in the opening.

6. The sensor assembly of claim 4, wherein the mechanical connector includes a rigid deformable clip.

7. The sensor assembly of claim 1, wherein the mechanical connector includes butterfly-style clip.

8. The sensor assembly of claim 7, wherein the image capture device is configured to automatically capture the one or more images in response to an actuation of the butterfly-style clip.

9. The sensor assembly of claim 1, wherein the mechanical connector includes arcuate jaws configured to be actuated by one or more buttons, each arcuate jaw connected at a first end to a hinge and including an inwardly facing surface configured to enclose around an exterior surface of the lumen and/or the syringe, the hinge configured to urge second ends of the arcuate jaws together, and the one or more buttons configured to cause the arcuate jaws to separate from each other in response to an actuation of the one or more buttons.

10. The sensor assembly of claim 9, wherein the image capture device is configured to automatically capture the one or more images in response to the actuation of the one or more buttons.

11. The sensor assembly of claim 1, wherein the at least one processor is configured to determine, based on the one or more images, the information associated with the medication delivery for the patient by:
detecting, in the one or more images, a patient identifier element associated with the patient; and
determining, based on the detected patient identifier element, patient information associated with the patient, wherein the patient information includes an indication of a type and/or a dosage of a medication to be delivered to the patient and/or a unique identifier of a medication delivery lumen to be used to deliver the medication to the patient.

12. The sensor assembly of claim 11, wherein the at least one processor is configured to determine, based on the one or more images, the information associated with the medication delivery for the patient by:
detecting, in the one or more images, a syringe identifier element associated with the syringe; and
determining, based on the detected syringe identifier element, syringe information associated with the syringe, wherein the syringe information includes a type and/or a dosage of a medication contained in the syringe.

13. The sensor assembly of claim 12, wherein the at least one processor is configured to determine, based on the one or more images, the information associated with the medication delivery for the patient by:
determining, based on the patient information and the syringe information, whether the medication contained in the syringe is a correct type and/or a correct dosage of the medication to be delivered to the patient; and
controlling, based on whether the medication contained in the syringe includes the correct type and/or the correct dosage of the medication to be delivered to the patient, the user feedback device to provide the audible and/or visual output to the user.

14. The sensor assembly of claim 13, wherein the at least one processor is configured to determine, based on the one or more images, the information associated with the medication delivery for the patient by:
detecting, in the one or more images, a lumen identifier element associated with the lumen; and
determining, based on the detected lumen identifier element, lumen information associated with the lumen, wherein the lumen information includes a unique identifier of the lumen.

15. The sensor assembly of claim 14, wherein the at least one processor is configured to determine, based on the one or more images, the information associated with the medication delivery for the patient by:
determining, based on the lumen information and the syringe information, whether the lumen is the medication delivery lumen to be used to deliver the medication to the patient; and
controlling, based on whether the lumen includes the medication delivery lumen to be used to deliver the medication to the patient, the user feedback device to provide the audible and/or visual output to the user.

16. The sensor assembly of claim 1, wherein the one or more images include a series of images including the syringe during the medication delivery for the patient, and wherein the at least one processor is configured to determine, based on the one or more images, the information associated with the medication delivery for the patient by:
detecting, in the one or more images, a movement of a stopper and/or a plunger rod of the syringe;
determining, based on the detected movement, at least one delivery parameter of the following delivery parameters: a patency of the lumen; an average rate of delivery of the medication; a current rate of the delivery of the medication; a steadiness index of the current rate of delivery of the medication; a volume of the medication delivered; a completion of the medication delivery for the patient; or any combination thereof; and
controlling, based on the at least one delivery parameter, the user feedback device to provide the audible and/or visual output to the user.

17. The sensor assembly of claim 1, wherein the one or more images include a series of images including one or more flush syringes connected to the lumen during a flush procedure, and wherein the least one processor is configured to determine, based on the one or more images, the information associated with the medication delivery for the patient by:
detecting, in the one or more images, a lumen identifier element associated with the lumen;
determining, based on the detected lumen identifier element, lumen information associated with the lumen, wherein the lumen information includes a unique identifier of the lumen;
detecting, in the one or more images, one or more flush syringe identifier elements associated with the one or more flush syringes;
determining, based on the one or more detected flush syringe identifier elements, flush syringe information associated with the one or more flush syringes, wherein the flush syringe information includes a number of flush syringes used for the flush procedure; and
determining, based on the lumen information and the flush syringe information, that the flush procedure has been performed for the lumen, a number of flush syringes used for the flush procedure, and/or a type of the flush procedure performed.

18. The sensor assembly of claim 1, further comprising:
wireless communications circuitry configured to wirelessly communicate with an external computing device,
wherein the at least one processor is configured to control the wireless communication circuitry to wirelessly communicate the information associated with the medication delivery for the patient to the external computing device.

19. A system, comprising:
an external computing device; and
one or more sensor assemblies, each sensor assembly of the one or more sensor assemblies including:
an image capture device configured to capture one or more images,
a user feedback device configured to provide an audible and/or visual output to a user, and
a mechanical connector configured to connect the sensor assembly to a lumen and/or a syringe configured to be connected to the lumen; and
a wireless communication link configured to facilitate communication between the external computing device,
wherein the external computing device includes a processor coupled to a memory and configured to, for each sensor assembly:
receive the one or more the images captured by the image capture device;
determine, based on the one or more images, information associated with a medication delivery for a patient; and
control, based on the information, via the wireless communication link, the user feedback device of the sensor assembly to provide the audible and/or visual output to the user.

20. A method, comprising:
capturing, with an image capture device of a sensor assembly, one or more images, wherein the sensor assembly includes a mechanical connector connecting the sensor assembly to a lumen and/or a syringe configured to be connected to the lumen;
obtaining, with at least one processor of the sensor assembly, the one or more the images captured by the image capture device;
determining, with the at least one processor of the sensor assembly, based on the one or more images, information associated with a medication delivery for a patient; and
controlling, with the at least one processor of the sensor assembly, based on the information, a user feedback device of the sensor assembly to provide an audible and/or visual output to the user.
